(19) United States
(12) Patent Application Publication
(10) Pub. No.: US 2012/0065152 A1
(43) Pub. Date: Mar. 15, 2012

(54) PRODRUGS OF GUANFACINE

(75) Inventors: Rhys Whomsley, Hampshire (GB); Bernard T. Golding, Newcastle upon Tyne (GB); Robert G. Tyson, Durham (GB)

(73) Assignee: SHIRE LLC, Florence, KY (US)

(21) Appl. No.: 13/232,502

(22) Filed: Sep. 14, 2011

Related U.S. Application Data
(60) Provisional application No. 61/383,056, filed on Sep. 15, 2010.

(30) Foreign Application Priority Data
Jan. 20, 2011 (GB) GB 1100981.8
Feb. 9, 2011 (GB) GB1102243.1

(51) Int. Cl. A61K 31/27; C07H 15/18; C07D 233/64; C07C 333/10; A61K 31/4164; A61P 1/00; A61P 25/00; A61P 25/30; A61P 17/04; A61P 25/22; A61P 9/12; A61P 29/00; C07C 279/24; A61K 31/7034

(52) U.S. Cl. 514/25; 560/24; 536/17.9; 560/32; 548/336.5; 558/240; 514/482; 514/400

(57) ABSTRACT

Prodrugs of guanfacine, pharmaceutical compositions containing such prodrugs and a method for providing therapeutic benefit in the treatment of ADHD/ODD (attention deficient hyperactivity disorder and oppositional defiance disorder) with guanfacine prodrugs are provided herein. Additionally, methods for improving the pharmacokinetics of guanfacine or minimizing or avoiding the adverse gastrointestinal side effects associated with guanfacine administration are provided herein.

### CROSS REFERENCE TO RELATED APPLICATIONS

This application claims the benefit of priority from U.S. Provisional Patent Application No. 61/383,056, filed Sep. 15, 2010, and UK Patent Application Serial Nos. GB 1100981.8 and GB 1102243.1 filed Jan. 20, 2011 and Feb. 9, 2011 respectively. The contents of each of which are incorporated herein by reference.

### FIELD OF THE INVENTION

The present invention relates to various prodrugs of guanfacine. In particular, the present invention relates to prodrugs of guanfacine which offer improved pharmacokinetic properties relative to guanfacine itself. The invention also relates to methods of reducing gastrointestinal (GI) side-effects associated with guanfacine therapy. These combined advantages should improve patient compliance and hence the drug's therapeutic effectiveness and patient benefit.

### BACKGROUND OF THE INVENTION

Attention Deficit Hyperactivity Disorder (ADHD) is one of the most common psychiatric conditions affecting children. Prevalence estimates vary but according to data from the National Survey of Children's Health, ˜8% of US children were diagnosed with ADHD in 2003, 56% of whom were treated with medication (Centers for Disease Control and Prevention (2005), Morb. Mortal. Wkly. Rep. 54, 842-847). Psychostimulant medications are the mainstay of therapy for patients with ADHD (Pediatrics (2001), 108, 1033-1044; Arch Gen Psychiatry (1999), 56, 1073-1085; Pediatrics (2004), 113, 754-761). Although >80% of these patients receive stimulant drugs, <40% are reported to exhibit normal behavior with treatment. Additionally, ˜30% of patients either do not respond or cannot tolerate long term therapy with these agents.

An additional concern is that these stimulants are classified by the US Drug Enforcement Administration as Schedule II Controlled Substances.

Several classes of non-stimulant drugs appear to be efficacious in patients with ADHD including tricylic antidepressants (imipramine and desipramine), bupropion, a norepinephrine and dopamine reuptake inhibitor, atomoxetine, a norepinephrine re-uptake inhibitor and a-2 adrenoceptor agonists clonidine and guanfacine. The latter has been reported to enhance frontal cortex functioning (PCF) in rats, monkeys and humans. In patients treated for ADHD with guanfacine, the drug may ameliorate prefrontal cortical deficits. Specifically, guanfacine appears to act primarily on the a-2 adrenoceptors in the prefrontal cortex, enhancing working memory, cognitive function and attentiveness.

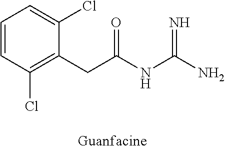

### Guanfacine: N-Amidino-2-(2,6-dichlorophenyl)acetamide monohydrochloride

Historically, guanfacine was employed as an antihypertensive agent (TENEX®) due to its effectiveness in lowering blood pressure. Typically, doses of 1-2 mg and occasionally 3 mg/day have been used in the treatment of hypertension. Peak plasma drug levels are reached as early as 1 hour after dosing and may be associated with cardiovascular side effects or somnolence. The drug is usually taken at night to minimize the impact of this. Recently a new guanfacine product (INTUNIV®) has been developed for the treatment of ADHD. This is a sustained release formulation designed to minimize any acute cardiovascular or CNS depressant effects of the drug resulting from the normally rapid rise in plasma drug concentrations. In a recent pharmacokinetic study on INTUNIV® reported by Swearingen et al. (2007), Clin. Therap. 29, 617-624, peak plasma levels were not seen until 6 hours post dosing so minimizing any unwanted cardiovascular or CNS effects.

In common with other a-2 adrenoceptor agonists such as clonidine, guanfacine may inhibit gut motility, leading, in some cases and especially after the higher doses, to constipation. For example, the incidence of constipation reported for the 3 mg dose of TENEX® is ˜15% (FDA label). This may be due in part to a direct local interaction between the drug and a-2 adrenoceptors within the gut. Published data provides evidence not only for the presence of a-2 adrenoceptors in the GI tract and their role in influencing gut motility (Blandizzi (2007), Neurochemistry International, 51, 282-288), but also for a direct effect of selective a-2 adrenoceptor agonists such as UK14,304 on the motility reflexes of guinea pig ileum (Stebbing et al (2001), J of Physiol. 534 465-478). Such effects are clearly undesirable.

INTUNIV® is a controlled release product and one limitation of such formulations is that they may be subject to a food interaction. The presence of food in the stomach serves to raise the gastric pH and slow gastric emptying. This may lead to some erosion of the enteric coating, designed to break down at higher pH's, and some early drug release as a consequence. Administration of INTUNIV® with a high fat meal has been shown to elevate Cₘₐₓ by 75% and increase AUC by 40% (FDA label). While taking the drug under more appropriate prandial conditions may be desirable, this may not always be possible. Variations in the prandial state may therefore lead to some variability in rate and extent of drug exposure. Previously, it has been demonstrated that the prandial state does not alter guanfacine pharmacokinetics following administration of both aqueous soluble and insoluble prodrugs to primates (see WO 2011/033296).

In spite of the advantages offered by guanfacine, there continues to be a need to reduce side-effects associated with guanfacine therapy. There remains therefore a real need in the treatment of ADHD as well as hypertension for a guanfacine product which retains all the inherent pharmacological advantages of the drug molecule but overcomes its limitations in inducing adverse cardiovascular, CNS and GI side-effects. The present invention addresses this need.

### SUMMARY OF THE INVENTION

In one aspect of the present invention, there is provided a guanfacine prodrug of Formula (I), or a pharmaceutically acceptable salt or tautomer thereof:

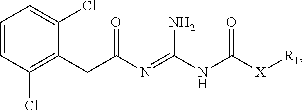

wherein

### X is O or S;

R₁ is a C₁₋₂₀ substituted or unsubstituted alkyl, glycosyl,

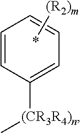

or a C₃₋₈ unsubstituted or substituted cycloalkyl;
R₂ is independently at each occurrence C₁₋₄ alkyl, C₁₋₄ alkoxy, halo, CN, NO₂, NH₂, SO₃H, OH, —CHO, —CO₂H,

### or —CH₂CO₂H;

n is 0, 1, 2 or 3;
m is 0, 1, 2, 3, 4 or, 5; and
R₃ and R₄ are each independently selected at each occurrence from the group comprising: hydrogen, hydroxy, —CO₂H, methyl, and —NH₂.

The combinations of the X, R₁, and R₂ groups contemplated within the scope of the present invention include those in which combinations of variables (and substituents) of the X, R₁, and R₂ groups are permissible so that such combinations result in stable compounds of Formula (I). For purposes of the present invention, it is understood that the combinations of the variables can be selected by one of ordinary skill in the art to provide compounds of Formula (I) that are chemically stable and that can be readily synthesized by techniques known in the art, as well as those methods set forth in the example section and figures.

In an embodiment, the guanfacine prodrug of the present invention is a conjugate containing a carbamate linkage.

In another aspect, the present invention provides a method of treating a disorder in a subject in need thereof with guanfacine. The method comprises orally administering an effective amount of a guanfacine prodrug of the present invention to the subject. The disorder may be one treatable with guanfacine. For example, the disorder may be attention deficit hyperactivity disorder (ADHD). An alternative psychiatric condition treatable with guanfacine is oppositional defiance disorder (ODD). Alternatively, the disorder may be a cardiovascular condition such as hypertension. The disorder may also be a disorder selected from the group consisting of: neuropathic pain, cognitive impairment associated with schizophrenia (CIAS), psychosis and working memory loss in the elderly, anxiety (including paediatric anxiety, PTSD, OCD, self injury), pruritis, addiction withdrawal and autism. The disorder may also be chemotherapy induced mucositis. The disorder may also be post traumatic stress syndrome. Alternatively, the disorder may be characterized by the patient suffering from hot flushes.

In another aspect, the present invention provides a guanfacine conjugate of the present invention for use in the treatment of attention deficit hyperactivity disorder (ADHD), oppositional defiance disorder (ODD), a cardiovascular condition such as hypertension, neuropathic pain, cognitive impairment associated with schizophrenia (CIAS), psychosis and working memory loss in the elderly, anxiety (including paediatric anxiety, PTSD, OCD, self injury), pruritis, addiction withdrawal, autism, chemotherapy induced mucositis, post traumatic stress syndrome or a disorder characterized by hot flushes.

In one embodiment, there is provided a method of reducing adverse gastrointestinal side effects associated with guanfacine treatment in a mammal. The method includes

(a) forming a guanfacine prodrug of Formula (I) or a pharmaceutically acceptable salt thereof; and
(b) administering the prodrug or a pharmaceutically acceptable salt thereof to a mammal in need thereof. Typically, the mammal is a human subject.

The guanfacine prodrugs described herein induce lower average (e.g., mean) effects on gut motility in the gastrointestinal environment as compared to a non-prodrug guanfacine salt form such as guanfacine HCl.

In an alternative aspect of the invention, a method for improving the pharmacokinetics and extending the duration of action of guanfacine in a subject in need thereof is provided. The method comprises administering to a subject in need thereof an effective amount of a prodrug of the present invention, or a composition thereof, wherein the plasma concentration time profile is modulated to minimize an initial upsurge in concentration of guanfacine, minimizing any unwanted cardiovascular or somnolent effects, while significantly extending the time for which the drug persists in plasma (resulting from continuing generation from the prodrug) and hence duration of action.

In a further aspect, a method for reducing inter- or intra-subject variability of guanfacine plasma levels is provided. The method comprises administering to a subject, or group of subjects in need thereof, an effective amount of a prodrug of the present invention, or a composition thereof.

In one preferred embodiment, the present invention is directed to a method for minimizing gastrointestinal side effects such as constipation normally associated with administration of guanfacine. The method comprises orally administering a guanfacine prodrug or pharmaceutically acceptable salt of the present invention, and wherein upon oral administration, the prodrug or pharmaceutically acceptable salt minimizes, if not completely avoids, the gastrointestinal side effects usually seen after oral administration of the unbound guanfacine. The amount of guanfacine is preferably a therapeutically effective amount.

The present invention relates to guanfacine prodrugs which preclude interaction between the a-2 adrenoceptors located in the gut and the active drug, so minimizing the risk of constipation. In addition, the prodrugs provided herein deliver a pharmacologically effective amount of the drug to treat various psychiatric and/or cardiovascular conditions. Such use of prodrugs of guanfacine may reduce intra- and inter-subject variability in plasma concentration and so provide consistent therapeutic efficacy. Additionally, the presence of quantities of unhydrolyzed prodrug in tissue compartments and/or plasma may provide a reservoir for continued generation of the active drug. Continued generation of guanfacine maintains plasma drug Levels, thereby reducing the frequency of drug dosage. These benefits would be expected to improve patient compliance.

These and other embodiments are disclosed or are apparent from and encompassed by the following Detailed Description.

### BRIEF DESCRIPTION OF THE DRAWINGS

FIG. 1 illustrates plasma concentration profiles for guanfacine following administration of guanfacine or guanfacine prodrug (compounds 3, 4, 5 and 6) to primates at 0.5 mg/kg guanfacine free base equivalents.

FIG. 2 illustrates plasma concentration profiles for guanfacine and guanfacine prodrug following administration of guanfacine prodrug (compound 3) to primates at 0.5 mg/kg guanfacine free base equivalents.

FIG. 3 illustrates analyte concentration profiles for guanfacine and guanfacine prodrug in rat tail vein following oral administration of guanfacine or guanfacine prodrug (compound 3) to rats at 1 mg/kg guanfacine free base equivalents.

FIG. 4 illustrates analyte concentration profiles for guanfacine and guanfacine prodrug in rat hepatic portal vein following oral administration of guanfacine prodrug (compound 3) to rats at 1 mg/kg guanfacine free base equivalents.

FIG. 5 illustrates guanfacine prodrug (compound 3) concentration profiles in rat hepatic portal vein following oral administration of guanfacine prodrug (compound 3) to rats at 1 mg/kg guanfacine free base equivalents.

FIG. 6 illustrates the effects of a guanfacine prodrug (compound 19) in the elevated plus maze model in rats at doses of 0.5 to 10 mg/kg. A—Effects of test substances on % of entries in open arms. B—Effects of test substances on time spent in open arms. For comparative purposes, the vehicle without any active was administered, as was guanfacine HCl and Clobazam. The number in parentheses is the dosage of the relevant compound.

### DETAILED DESCRIPTION OF THE INVENTION

### A. Definitions

As used herein:

The term “alkyl,” as a group, refers to a straight or branched hydrocarbon chain containing the specified number of carbon atoms. When the term “alkyl” is used without reference to a number of carbon atoms, it is to be understood to refer to a C₁-C₂₀ alkyl group, preferably a C₁-C₁₀ alkyl group. For example, C₁₋₁₀ alkyl refers to a straight or branched alkyl containing at least 1, and at most 10, carbon atoms. For another example, C₂₋₇ alkyl refers to a straight or branched alkyl containing at least 2, and at most 7, carbon atoms. Examples of “alkyl” as used herein include, but are not limited to, methyl, ethyl, n-propyl, n-butyl, n-pentyl, i-butyl, i-propyl, t-butyl, hexyl, heptyl, octyl, nonyl and decyl. Preferably, the alkyl group is a lower alkyl of from about 1 to 7 carbons, yet more preferably about 1 to 4 carbons. The alkyl group can be substituted or unsubstituted.

The term “substituted alkyl” as used herein denotes alkyl radicals wherein at least one hydrogen is replaced by one or more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “carbonyl” refers to a group —C(═O).

The term “carboxyl” refers to a group —CO₂H and consists of a carbonyl and a hydroxyl group (More specifically, C(═O)OH).

The term “substituted” refers to adding or replacing one or more atoms contained within a functional group or compound with one of the moieties from the group of halo, oxy, azido, nitro, cyano, alkyl, alkoxy, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl carboxyl, and amino groups.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “halo” or “halogen” refers to fluoro, chloro, bromo, and iodo.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. In some embodiments, water or aqueous solution-based formulations are employed as carriers for orally administered formulations. In other embodiments, oil-based formulations are employed as carriers for orally-administered formulations. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in humans.

A “pharmaceutically acceptable excipient” means an excipient that is useful in preparing a pharmaceutical composition that is generally safe, non-toxic and neither biologically nor otherwise undesirable, and includes an excipient that is acceptable for human pharmaceutical use. A “pharmaceutically acceptable excipient” as used in the present application includes both one and more than one such excipient.

The term “treating” includes: (1) preventing or preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (2) reducing or inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

The term “subject” refers to humans.

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be amelioration of the typical symptoms of ADHD. In further and/or alternative embodiments, the therapeutic response will be amelioration of the typical symptoms of opposition defiance disorder (ODD), hypertension, pain (neuropathic pain), cognitive impairment in psychosis, cognitive impairment associated with schizophrenia (CIAS), psychosis and working memory loss in the elderly, post traumatic stress disorder (PTSD), anxiety (including paediatric anxiety, PTSD, OCD, self injury), addiction withdrawal, autism, hot flushes, pruritic, chemotherapy-induced mucositis, etc. It is further within the competency of one skilled in the art to determine appropriate treatment duration, appropriate doses, and any potential combination treatments, based upon an evaluation of therapeutic response.

“Reducing gastrointestinal side effects associated with guanfacine therapy” shall be understood to mean a reduction, amelioration and/or prevention and/or prevention of the occurrence of gastrointestinal side effects (e.g., constipation) realized in patients treated with the prodrug described herein as compared to patients which have received a non-prodrug guanfacine salt in an immediate release or sustained release form. Reduction of gastrointestinal side effects is deemed to occur when a patient achieves positive clinical results. For example, successful reduction of gastrointestinal side effects shall be deemed to occur when at least about 10% (i.e. at least about 15%) or preferably at least about 20%, more preferably at least about 30% or higher (i.e., about 40%, 50%) decrease in constipation including other clinical markers contemplated by the artisan in the field is realized when compared to that observed in the treatment with a non-prodrug guanfacine. In certain aspects, successful reduction of gastrointestinal side effects can be determined by changes in gut motility induced by the prodrug described herein as compared to a non-prodrug guanfacine salt in an immediate release or sustained release form. In this aspect, statistical significance relative to a non-prodrug guanfacine can be at least about 0.058, and preferably <0.001.

The term “at least about” comprises the numbers equal to or larger than the numbers referred to. In various embodiments, such as when referring to the decrease in gut motility, the term “at least about 15%” includes the terms “at least about 16%”, “at least about 17%”, at least about 18%” and so forth. Likewise, in some embodiments, the term “at least about 30%” includes the terms “at least about 31%”, “at least about 32%”, and so forth.

The term “active ingredient,” unless specifically indicated, is to be understood as referring to the guanfacine portion of the prodrug, as described herein.

The term “salts” can include acid addition salts or addition salts of free bases. Suitable pharmaceutically acceptable salts include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate (see, for example, Berge, et al. “Pharmaceutical Salts,” J. Pharm. Sci. 1977; 66:1).

The term “about,” unless otherwise indicated, refers to ±10% of the given value.

The present invention also includes the synthesis of all pharmaceutically acceptable isotopically-labelled compounds of Formula (I) wherein one or more atoms are replaced by atoms having the same atomic number, but an atomic mass or mass number different from the atomic mass or mass number most commonly found in nature.

Substitution with stable isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labelled compounds can generally be prepared by conventional techniques known to those skilled in the art or by processes analogous to those described using an appropriate isotopically-labelled reagent in place of the non-labelled reagent previously employed.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### B. Compounds of Formula (I)

In one aspect of the present invention as defined above, there is provided a guanfacine prodrug of Formula (I), or a pharmaceutically acceptable salt or tautomer thereof:

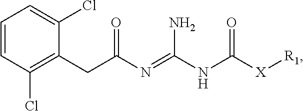

wherein

### X is or S;

R₁ is a C₁₋₂₀ substituted or unsubstituted alkyl, glycosyl,

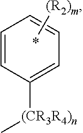

or a C₃₋₈ unsubstituted or substituted cycloalkyl;
R₂ is independently at each occurrence C₁₋₄ alkyl, C₁₋₄ alkoxy, halo, CN, NO₂, NH₂, SO₃H, OH, —CHO, —CO₂H, or —CH₂CO₂H;
n is 0, 1, 2 or 3;
m is 0, 1, 2, 3, 4 or, 5; and
R₃ and R₄ are each independently selected at each occurrence from the group comprising: hydrogen, hydroxy, —CO₂H, methyl, and —NH₂.

In an embodiment:

### X is O or S;

R₁ is a C₁₋₇ substituted or unsubstituted alkyl, glycosyl,

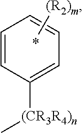

or a C₃₋₈ unsubstituted or substituted cycloalkyl;
R₂ is independently at each occurrence C₁₋₄ alkyl, C₁₋₄ alkoxy, halo, CN, NO₂, NH₂, SO₃1-1, OH, —CHO, —CO₂H, or —CH₂CO₂H;
n is 0, 1, 2 or 3;
m is 0, 1, 2, 3, 4 or, 5; and
R₃ and R₄ are each independently selected at each occurrence from the group comprising: hydrogen, hydroxy, —CO₂H, methyl, and —NH₂.

In an embodiment, X is O.

In an alternate embodiment, X is S.

In an embodiment R₂ is independently at each occurrence OH, —CHO, —CO₂H, or —CH₂CO₂H.

In an embodiment X is O and R₂ is independently at each occurrence OH, —CHO, —CO₂H, or —CH₂CO₂H.

In an embodiment, R₁ is a substituted or unsubstituted C₁₋₇ alkyl. In an embodiment, R₁ is a C₁₋₇ alkyl substituted with one or two groups selected from the group consisting of: hydroxy, alkoxy, amino, aryl, heteroaryl and halo. In an embodiment, R₁ is a C₁₋₇ alkyl substituted with an amino group and a heteroaryl group. In an embodiment, R₁ is

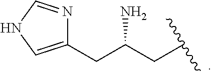

In an embodiment, R₁ is a C₂₋₇ alkyl.

In an embodiment, R₁ is a C₂₋₆ alkyl. For example, R₁ is ethyl, n-propyl, isopropyl, n-butyl, sec-butyl, isobutyl, t-butyl or neopentyl. In an alternate example, R₁ is ethyl, n-propyl, isopropyl, n-butyl, sec-butyl, isobutyl or neopentyl. Preferably R₁ is ethyl.

In an embodiment, R₁ is a C₆ alkyl. For example, R₁ is hexyl.

In an embodiment R₁ contains one or more deuterium atoms.

In an embodiment, X is O and R₁ is a C₂₋₅ alkyl, preferably ethyl. For example, X is O and R₁ is ethyl, n-propyl, isopropyl, n-butyl, sec-butyl, isobutyl, t-butyl or neopentyl. In an alternate example, X is O and R₁ is ethyl, n-propyl, isopropyl, n-butyl, sec-butyl, isobutyl or neopentyl.

In an alternate embodiment, X is S and R₁ is a C₂₋₆ alkyl, preferably ethyl. For example, X is S and R₁ is ethyl, n-propyl, isopropyl, n-butyl, sec-butyl, isobutyl, t-butyl or neopentyl. In an alternate example, X is S and R₁ is ethyl, n-propyl, isopropyl, n-butyl, sec-butyl, isobutyl or neopentyl.

In an embodiment, R₁ is a substituted C₁alkyl. For example, R₁ is carboxylmethyl.

In an embodiment, R₁ is a cycloalkyl substituted C₁alkyl. For example R₁ is cyclopropyl methyl.

In an embodiment, X is O and R₁ is a substituted C₁alkyl.

In an embodiment, X is O and R₁ is a cycloalkyl substituted C₁ alkyl.

In an embodiment, R₁ is a substituted or unsubstituted cycloalkyl.

In an embodiment R₁ is a substituted or unsubstituted cyclohexyl. For example, R₁ is menthyl.

In an embodiment R₁ is glycosyl. Where R₁ is glycosyl, the carbohydrate moiety is linked to the guanfacine portion of the prodrug using any suitable hydroxyl group. In a particular embodiment, R₁ is a hexose. Preferably R₁ is glucose.

In an embodiment, X is O and R₁ is a substituted or unsubstituted cycloalkyl.

In an embodiment, X is O and R₁ is a substituted or unsubstituted cyclohexyl.

In an embodiment X is O and R₁ is glycosyl. In a particular embodiment, X is O and R₁ is a hexose. Preferably X is O and R₁ is glucose.

In an alternate embodiment, R₁ is a C₂₋₄ hydroxyalkyl. For example, R₁ is 2-hydroxyethyl or 3-hydroxypropyl.

In an alternate embodiment, X is O and R₁ is a C₂₋₄ hydroxyalkyl.

In an embodiment, R₁ is

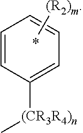

In an embodiment, R₁ is

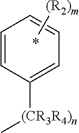

and m is 1.

In an embodiment, R₁ is

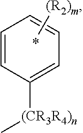

n is 0 and m is 0.

In an embodiment R₁ is

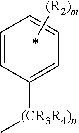

### m is 1, and R₂ is OH, —CHO, —CO₂H, or —CH₂CO₂H.

In an embodiment R₁ is

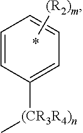

### n is 0, m is 1 and R₂ is OH, —CHO, —CO₂H, or —CH₂CO₂H.

In an embodiment R₁ is

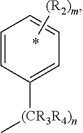

m is 1, 2, 3, 4, or 5 and at least one R₂ is OH.

In an embodiment R1 is

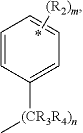

n is 0, m is 1, 2, 3, 4, or 5 and at least one R₂ is OH.

In an embodiment, R₁ is

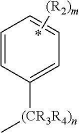

and n is 0.

In an embodiment, R₁ is

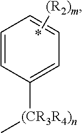

### n is 0, m is 1 and R₂ is —CO₂H, or —CH₂CO₂H.

In an embodiment, R₁ is

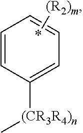

### n is 0, m is 1 and R₂ is —CH₂CO₂H.

In an embodiment, R₁ is

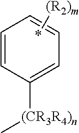

### n is 0, m is 1 and R₂ is —CO₂H.

In an embodiment, X is O and R₁ is

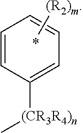

In an embodiment, X is O, R₁ is

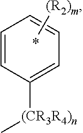

### n is 0, in is 1 and R₂ is —CO₂H, or —CH₂CO₂H.

In an embodiment, X is O, R₁ is

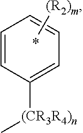

### n is 0, in is 1 and R₂ is CH₂CO₂H.

In an embodiment, X is O, R₁ is

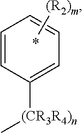

### n is 0, in is 1 and R₂ is —CCO₂H.

In an embodiment, R₁ is

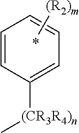

and n is 1, preferably wherein R₃ and R₄ are H.

In an embodiment, R₁ is

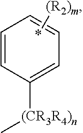

### n is 1, m is 0, R₃ and R₄ are H.

In an embodiment X is O, R₁ is

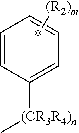

and n is 1, preferably wherein R₃ and R₄ are H.

In an embodiment X is O, R₁ is

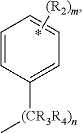

### n is 1, m is O, R₃ and R₄ are H.

In a particular embodiment, the compounds of Formula (I) include:

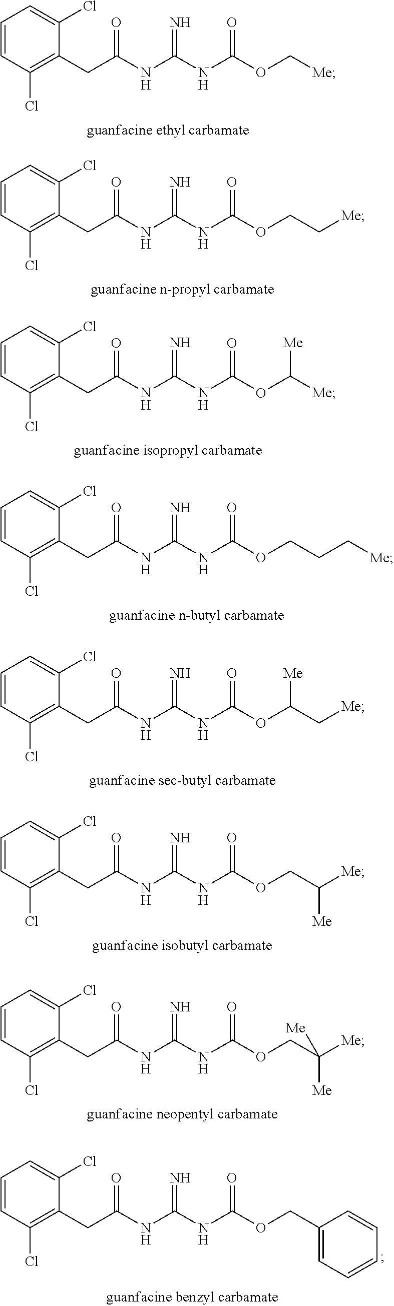

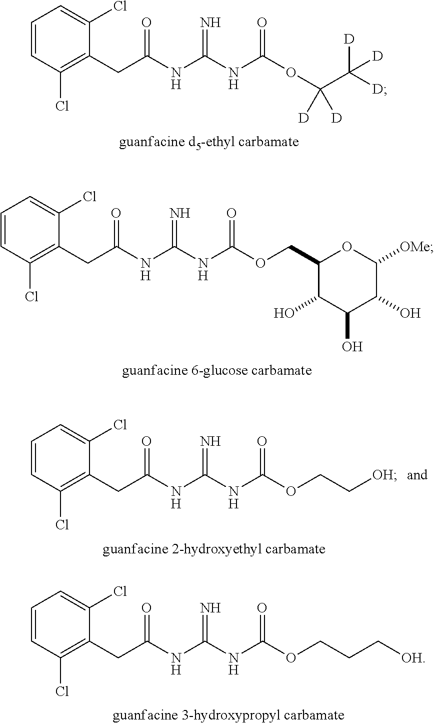

In a particular embodiment the compounds of formula (I) include

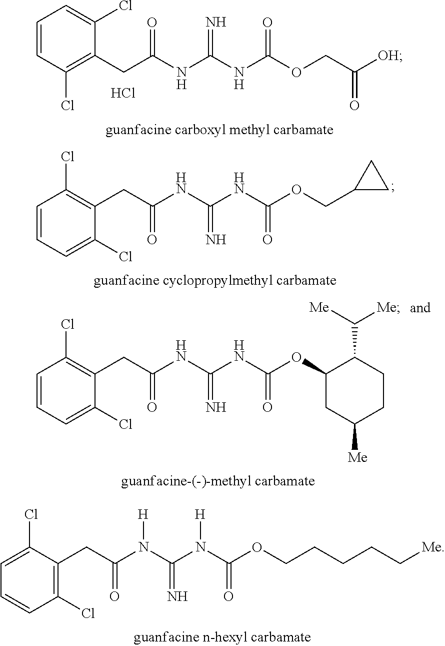

In another particular embodiment, the compounds of Formula (I) include:

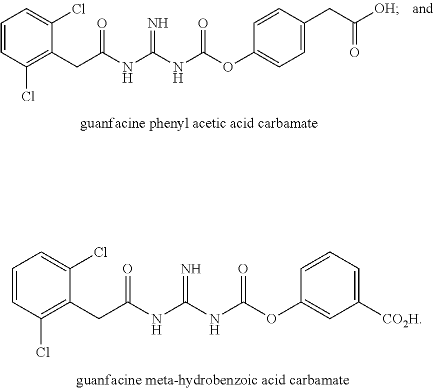

In an embodiment, X together with R₁ is not

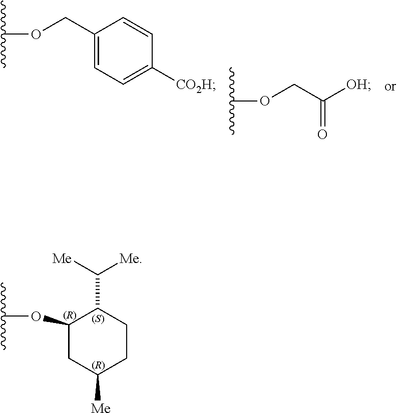

In an embodiment, the invention does not provide compounds of formula I wherein X is O and R₁ is a substituted C₂₋₇ group which is substituted with M-OH; wherein, M is absent or is selected from the group consisting of:

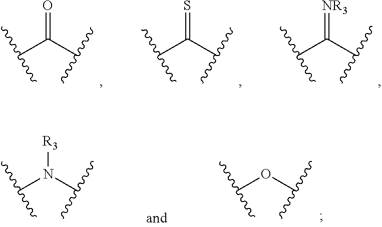

wherein R³ is selected from the group consisting of: H, C₁₋₄ alkyl and C₃₋₈ cycloalkyl.

### C. Advantages of the Guanfacine Prodrugs of the Present Invention

The use of the guanfacine prodrugs of the present invention provides a means of delaying the Tₘₐₓ compared to the use of IR guanfacine to minimize the impact of Cₘₐₓ related side effects. The slower dissolution of the prodrugs compared to the active drug allows a more gradual intestinal absorption.

Once absorbed, these prodrugs may provide a reservoir from which the active drug species may continue to be generated simulating the delivery from a sustained release preparation. This approach avoids the need for enteric coated sustained release formulations which may be subject to premature coat erosion in the stomach due to the presence of food.

A further advantage of the invention is that it enables prodrug compounds to be obtained in relatively high purity and essentially free of guanfacine itself. In other words, the prodrugs can be produced with minimal or no free guanfacine being present. Thus, the prodrugs of the invention are able to avoid any unwanted local effect following dosing which would otherwise be due to guanfacine itself. After absorption, the prodrugs can then provide, following cleavage, guanfacine which is available to provide its therapeutic effect without having initially given rise to any significant local effects.

The use of the guanfacine prodrugs of the present invention provides a means of delivering guanfacine to the systemic circulation but avoiding direct contact between the active drug and a-2-adrenoceptors in the GI tract so minimizing any potential constipating effects. It is possible that part of the constipating actions of a-2-adrenoceptors may be elicited directly within the gut. Reduction of the adverse GI side-effects associated with administration may be a particular advantage of using a prodrug of the present invention.

Preferably, guanfacine therapy with the prodrugs described herein, when administered orally, induces significantly lower average (i.e. mean) effects on gut motility in the gastrointestinal environment of the patient than a non-prodrug guanfacine salt form such as guanfacine hydrochloride salt.

Additionally, the use of the prodrugs of the present invention can provide greater consistency in response as the result of more consistent oral bioavailability. As a result of this consistent oral bioavailability, the prodrugs of the present invention offer a significant reduction of inter- and intrasubject variability of guanfacine plasma and CNS concentrations and, hence, significantly less fluctuation in therapeutic response for a single patient, or among a patient population providing improved patient benefit.

### D. Methods of Treatment

The present invention provides a method for treating a disorder in a subject in need thereof with guanfacine. The method comprises orally administering an effective amount of a guanfacine prodrug of the present invention to the subject. The disorder may be one treatable with guanfacine. For example, the disorder may be psychiatric conditions such as attention deficit hyperactivity disorder or oppositional defiance disorder. The prodrug can be any guanfacine prodrug encompassed by Formula (I).

The present invention also provides a guanfacine conjugate of Formula (I) for use in the treatment of a psychiatric condition such as attention deficit hyperactivity disorder or oppositional defiance disorder.

In one aspect, the present invention is directed to a method for minimizing the gastrointestinal side effects normally associated with administration of guanfacine. The method comprises orally administering a guanfacine prodrug or pharmaceutically acceptable salt of the present invention, and wherein upon oral administration, the prodrug or pharmaceutically acceptable salt minimizes, if not completely avoids, the constipating effects frequently seen after administration of higher oral doses of the unbound guanfacine. The amount of guanfacine is preferably a therapeutically effective amount. The prodrug can be any guanfacine prodrug encompassed by Formula (I).

In view of the above, there are provided methods of reducing gastrointestinal side effects associated with guanfacine therapy in a mammal. The methods include:

(a) forming a guanfacine prodrug of Formula (I) or a pharmaceutically acceptable salt thereof; and

(b) administering the prodrug or a pharmaceutically acceptable salt thereof to a mammal in need thereof.

In another aspect, the invention provides a method of treating an attention deficit hyperactivity disorder in a mammal. The method includes administering a prodrug of Formula (I) or a pharmaceutically acceptable salt thereof to a mammal in need thereof.

The present invention also provides a guanfacine conjugate of Formula (I) for use in the treatment of attention deficit hyperactivity disorder in a mammal.

In yet another aspect, the invention provides a method of treating hypertension in a mammal. The method is conducted by administering a prodrug of Formula (I) or a pharmaceutically acceptable salt thereof to a mammal in need thereof.

The present invention also provides a guanfacine conjugate of Formula (I) for use in the treatment of hypertension in a mammal.

Ideally, the prodrug employed in the methods described herein, when administered orally, should achieve therapeutically effective guanfacine plasma concentrations.

In one preferred embodiment, the prodrugs of Formula (I) or the pharmaceutically acceptable salts thereof are orally administered. In some preferred embodiments, the method protocol includes administering the prodrugs of Formula (I) or the pharmaceutically acceptable salts thereof in a daily amount of from about 1 mg to about 100 mg, preferably from about 1 mg to about 50 mg, more preferably from about 1 mg to about 15 mg, more preferably from about 1 mg to about 10 mg and more preferably from about 1 mg to about 5 mg based on the amount of guanfacine in free base form. If the systemic availability from the prodrug yields a lower absolute oral bioavailablity, then the preferred dosage is from about 2 mg to about 10 mg.

In all aspects of the invention where the conjugate of Formula (I) or the pharmaceutically acceptable salt thereof is administered, the dosage mentioned is based on the amount of guanfacine free base rather than the amount of the conjugate administered.

The present method is useful for, among other things, avoiding the constipating effects associated with guanfacine administration resulting from a-2a adrenoceptor mediated inhibition of gut motility as compared to a treatment with guanfacine in non-prodrug salt form.

Alternatively, the present invention provides a method for improving the pharmacokinetics of guanfacine in a subject in need thereof. The method comprises administering to a subject in need thereof an effective amount of a prodrug of the present invention, or a composition thereof, wherein the rate and consistency of delivery of guanfacine provided by the prodrug offers advantage over that seen when guanfacine in a non-prodrug form is administered alone. These benefits include a modulation of the attainment of Cₘₐₓ so minimizing unwanted cardiovascular effects, greater consistency in attainment of plasma levels and thereby therapeutic response and prolonged maintenance of plasma drug levels reducing dosing frequency and improving patient compliance. The prodrug can be any guanfacine prodrug encompassed by Formula (I).

In a further alternative aspect, the present invention provides a method of reducing effects of guanfacine on gut motility. The method includes the steps of

(a) reacting guanfacine with an activated alcohol capable of forming a covalent bond with the guanfacine under conditions effective to form a prodrug of Formula (I) and
(b) administering the prodrug of Formula (I) or the pharmaceutically acceptable salt thereof to a mammal in need thereof.

The present invention also provides a guanfacine conjugate of Formula (I) for use in the reduction of the effects of guanfacine on gut motility.

### E. Salts, Solvates, & Derivatives of the Compounds of the Invention

The methods of the present invention further encompass the use of salts and solvates of the guanfacine prodrugs described herein. In one embodiment, the invention disclosed herein is meant to encompass all pharmaceutically acceptable salts of guanfacine prodrugs.

Typically, a pharmaceutically acceptable salt of a prodrug of guanfacine used in the practice of the present invention is prepared by reaction of the prodrug with an acid as appropriate. The salt may precipitate from solution and be collected by filtration or may be recovered by evaporation of the solvent in accordance with methods well known to those skilled in the art.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metal bases or amines, such as alkali and alkaline earth metal hydroxides or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, for example, Brittain. Polymorphism in Pharmaceutical solids. Marcel Decker, New York, 1999.). The compounds useful in the practice of the present invention can have one or more chiral centers and, depending on the nature of individual components, they can also have geometrical isomers.

### F. Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier or excipient selected with regard to the intended route of administration and standard pharmaceutical practice. The compositions of the present invention also include pharmaceutically acceptable salts of the guanfacine prodrugs, as described above.

While it is anticipated that the formulations of the invention may be immediate-release dosage forms, i.e., dosage forms that release the prodrug at the site of absorption immediately, in an alternative embodiment, the prodrugs described herein can be as part of controlled-release formulation, i.e. dosage forms that release the prodrug over a predetermined period of time. Controlled release dosage forms may be of any conventional type, e.g. in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20^{th} Edition, 2000, pp. 858-914.

For those prodrugs of guanfacine which do not result in sustained plasma drugs levels due to continuous generation of active from a systemic reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption takes place and effectively prolonging plasma drug levels. Other drug delivery systems affording delayed progression along the GI tract may also be of value.

The formulations of the present invention can be administered from one to six times daily, depending on the dosage form and dosage.

In one aspect, the present invention provides a pharmaceutical composition containing at least one active pharmaceutical ingredient (i.e., a guanfacine prodrug), or a pharmaceutically acceptable derivative (e.g., a salt or solvate) thereof, and a pharmaceutically acceptable carrier or other excipient. In particular, the invention provides a pharmaceutical composition including a therapeutically effective amount of at least one prodrug described herein, or a pharmaceutically acceptable derivative thereof, and a pharmaceutically acceptable carrier or excipient.

For the methods of the invention, the prodrug employed in the present invention may be used in combination with other therapies and/or active agents. Accordingly, the present invention provides, in a further aspect, a pharmaceutical composition including at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds must be stable and compatible with each other and the other components of the formulation. When formulated separately the compounds may be provided in any convenient formulation, conveniently in such manner as is known for such compounds in the art.

The prodrugs used herein may be formulated for administration in any convenient way for use in human medicine and the invention therefore includes within its scope pharmaceutical compositions comprising a compound of the invention adapted for use in human medicine. Such compositions may be presented for use in a conventional manner with the aid of one or more pharmaceutically acceptable excipients or carriers. Acceptable carriers and excipients for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may include, in addition to the carrier, any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example, see International Patent Application No. WO 02/00196 (SmithKline Beecham).

The prodrugs and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, and propyl paraben).

Suitable examples of pharmaceutically acceptable stabilizers and antioxidants include, but are not limited to, ethylenediaminetetra-acetic acid (EDTA), thiourea, tocopherol and butyl hydroxyan (hydroxyanisole).

The pharmaceutical compositions of the invention may contain from 0.01 to 99% weight per volume of the prodrugs encompassed by the present invention.

### G. Doses

The doses described throughout the specification refer to the amount of guanfacine in the composition, in free base form.

Appropriate patients (subjects) to be treated according to the methods of the invention include any human in need of such treatment. Methods for the diagnosis and clinical evaluation of ADHD or ODD including the severity of the condition experienced by a human are well known in the art. Thus, it is within the skill of the ordinary practitioner in the art (e.g., a medical doctor) to determine if a patient is in need of treatment.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

In a preferred embodiment, an effective amount of prodrugs of Formula (I) is from about 1 mg to about 100 mg, preferably from about 1 to about 50 mg, and more preferably from about 1 mg to about 5 mg. If the prodrugs of Formula (I) provide near complete oral bioavailability, the preferred dosage is from about 1 to about 5 mg, based on the currently effective maximum daily doses of from about 1 to about 5 mg. If the systemic availability from the prodrug yields a lower absolute oral bioavailablity, then the preferred dosage is from about 2 mg to about 10 mg. The prodrugs, as described herein, may be administered once daily or divided into multiple doses as part of multiple dosing treatment protocol. In all aspects of the invention where guanfacine prodrugs are administered, the dosage amount mentioned is based on the amount of guanfacine in free base form.

Depending on the severity of the condition to be treated, a suitable therapeutically effective and safe dosage, as may readily be determined within the skill of the art, and without undue experimentation, may be administered to subjects. For oral administration to humans, the daily dosage level of the prodrug may be in single or divided doses. The duration of treatment may be determined by one of ordinary skill in the art, and should reflect the magnitude of the condition.

In the methods of treating ADHD/ODD or hypertension, the prodrugs encompassed by the present invention may be administered in conjunction with other therapies and/or in combination with other active agents. For example, the prodrugs encompassed by the present invention may be administered to a patient in combination with other active agents used in the management of these conditions. An active agent to be administered in combination with the prodrugs encompassed by the present invention may include, for example, a drug selected from the group consisting of stimulant drugs such as amphetamine or methyl phenidate or non stimulant agents such atomoxetine. In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating ADHD/ODD or hypertension, the dose of each compound may differ from that when the compound is used alone. Appropriate doses will be readily appreciated by those skilled in the art.

### H. Synthesis of the Prodrugs

Generally, the methods of preparing prodrugs of Formula (I) include reacting guanfacine with an activated alcohol under conditions effective to form prodrugs of Formula (I). Activated alcohols useful in the methods described herein can be prepared by standard techniques known to those of ordinary skill, for example, reacting an alcohol (or thiol) with oxalyl chloride to form a chloroformate or with DSC to prepare an activated carbonate. The methods provide a guanfacine prodrug where guanfacine is bonded to an alcohol through a carbamate or thiomarbamate linkage.

For purposes of illustration, the methods of preparing prodrugs described herein include: (a) reacting an activated alcohol or thiol having the formula;

LG-C(═O)—X—R₁

with guanfacine under basic conditions sufficient to form a prodrug of the formula (I):

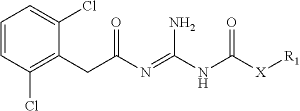

wherein
LG is a leaving group;

### X is O or S;

R₁ is a C₁₋₇ substituted or unsubstituted alkyl, glycosyl,

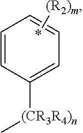

or a C₃₋₈ unsubstituted or substituted cycloalkyl;
R₂ is independently at each occurrence C₁₋₄ alkyl, C₁₋₄ alkoxy, halo, CN, NO₂, NH₂, SO₃H, OH, —CHO, —CO₂H, or —CH₂CO₂H;
n is 0, 1, 2 or 3;
m is 0, 1, 2, 3, 4 or, 5; and
R₃ and R₄ are each independently selected at each occurrence from the group comprising: hydrogen, hydroxy, —CO₂H, methyl, and —NH₂.

The leaving group useful in the preparation includes halogen, NHS or p-nitrophenyloxy and other leaving groups known by those of ordinary skill in the art.

It will be understood that other art recognized protecting groups can be used in place of BOC and t-Bu.

Preferably, the reactions are carried out in an inert solvent such as 1,2-dimethoxyethane (DME), ethyl acetate, methanol, methylene chloride, chloroform, THF, N,N′-dimethylformamide (DMF) or mixtures thereof. The reactions can be preferably conducted in the presence of a base, such as N-methylmorpholine (NMM), dimethylaminopyridine (DMAP), diisopropylethylamine, pyridine, triethylamine, etc. to neutralize any acids generated. The reactions can be carried out at a temperature from about 0° C. up to about 22° C. (room temperature).

Alternatively, compounds of Formula (I) can be prepared without undue experimentation by using standard techniques known to those of ordinary skill in the field.

### EXAMPLES

Preferably the present invention is further illustrated by reference to the following Examples. However, it should be noted that these Examples, like the embodiments described above, are illustrative and are not to be construed as restricting the enabled scope of the invention in any way. The bold-faced numbers recited in the Examples correspond to those shown in the reaction schemes. Abbreviations are used throughout the examples such as, DCC (dicyclohexylcarbodlimide), NMM (N-methylmorpholine), DME (1,2-dimethoxyethane), NHS (N-hydroxysuccinimide), TFA (trifluoroacetic acid), DSC (N,N′-disuccinimidyl carbonate), THF (tetrahydrofuran) and DMF (N,N′-dimethylformamide).

### Example 1

### Synthesis of Compounds of Formula (I)

The synthesis of alkyl guanfacine carbamate was achieved as shown in Scheme 1 by reacting guanfacine HCl with alkyl chloroformate in the presence of NMM in THF. The synthetic route is shown below in Scheme 1.

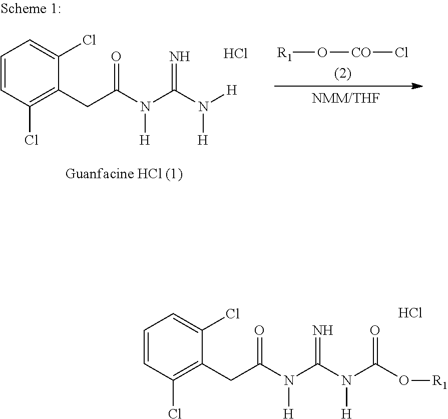

### Example 2

### Preparation of Guanfacine Carbamate Prodrugs

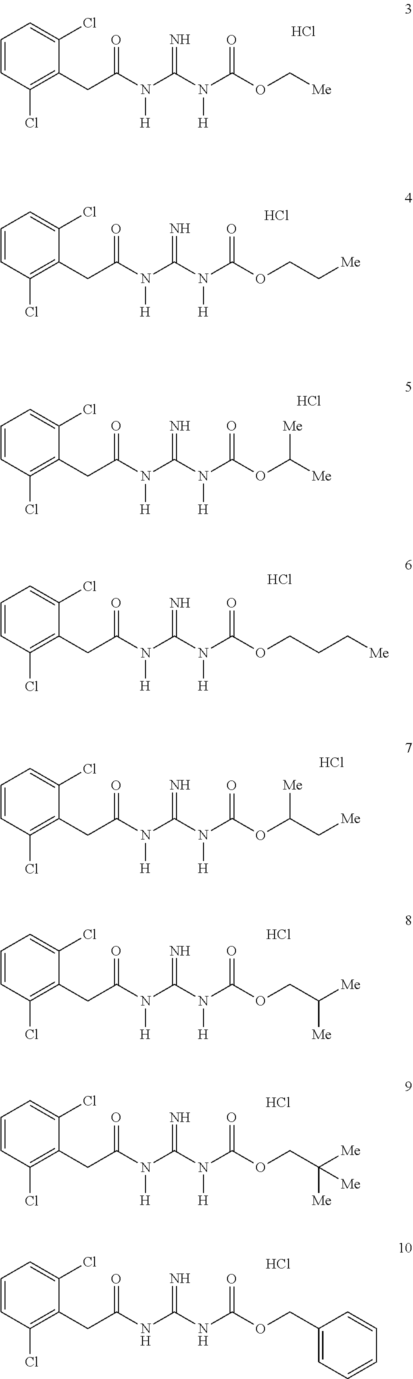

### Compound 3: {N′-[2-(2,6-Dichloro-phenyl)-acetyl]-guanidinocarbonyloxy}ethane Hydrochloride

Trivial name: Guanfacine ethyl carbamate Hydrochloride

Appearance: white solid; LCMS: m/z=317.85 consistent for protonated ion (MH⁺); ¹H NMR (DMSO-d₆): 8.83 (br, 2H, NH₂⁺), 7.49 (d, J=7.5 Hz, 2H, 2×ArH), 7.35 (m, 1H, ArH), 4.01 (m, 4H, ArCH₂CH₂), 1.18 (t, J=6.9 Hz 3H, CH₃); Purity: >99% by HPLC, no free guanfacine content by HPLC; Solubility: >10 mg/mL in DMSO, <1 mg/mL in water.

### Compound 4: 1-{N′-[2-(2,6-Dichloro-phenyl)acetyl]-guanidinocarbonyloxy}propane Hydrochloride

Trivial name: Guanfacine n-propyl carbamate Hydrochloride

To a stirred suspension of guanfacine hydrochloride (2.83 g, 10.02 mmol) and 4-methylmorpholine (1.01 g, 1.10 mL, 10.02 mmol) in dry THF (60 mL), under an atmosphere of nitrogen, was added n-propyl chloroformate (1.23 g, 1.13 mL, 10.02 mmol) and stirring was continued at room temperature overnight. The mixture was filtered and the filtrate was concentrated to yield a white solid. The residue was purified using a Biotage Isclera automated chromatography system under normal phase conditions (silica column, gradient of 10→100% EtOAc in petrol) with detection at 254 nm to afford guanfacine n-propyl carbamate hydrochloride (970 mg, 26%), as a white solid.

Appearance: white solid; LCMS: m/z=331.90, consistent for protonated parent ion (MH⁺); ¹H NMR (DMSO-d₆): 11.27 (br, 1H, NH), 8.83 (br, 2H, NH₂⁺), 7.49 (d, J=8.3 Hz, 2H, 2×ArH), 7.35 (m, 1H, ArH), 4.06 (s, 2H, ArCH₂), 3.94 (t, J=6.5 Hz, 2H, CH₂), 1.58 (m, 2H, CH₂), 0.89 (t, J=7.4 Hz, 3H, CH₃); Purity: >95% by HPLC, no free guanfacine by HPLC; Solubility: >10 mg/mL in DMSO, <1 mg/mL in water, >1 mg/mL in water/CH₃CN (1:1).

### Compound 5: 2-{N′-[2-(2,6-Dichloro-phenyl)acetyl]-guanidinocarbonyloxy}propane Hydrochloride

Trivial name: Guanfacine isopropyl carbamate Hydrochloride

Appearance: white solid; LCMS: m/z=331.90, consistent for protonated parent ion (MH⁺); ¹H NMR (DMSO-d₆): 8.80 (br, 2H, NH₂′), 7.49 (d, J=7.8 Hz, 2H, 2×ArH), 7.35 (m, 1H, ArH), 4.77 (m, 1H, CH), 4.05 (s, 2H, ArCH₂), 1.18 (d, J=6.3 Hz, 6H, 2×CH₃); Purity: >95% by HPLC, no free guanfacine by HPLC; Solubility: >10 mg/mL in DMSO, <1 mg/mL in water, >5 mg in 3.0 mL CH₃CN/3.0 mL H₂O.

### Compound 6: 1-{N′-[2-(2,6-Dichloro-phenyl)-acetyl]-guanidinocarbonyloxy}butane Hydrochloride

Trivial name: Guanfacine n-butyl carbamate Hydrochloride

To a stirred suspension of guanfacine hydrochloride (2.83 g, 10.02 mmol) and 4-methylmorpholine (1.01 g, 1.10 mL, 10.02 mmol) in dry THF (60 mL), under an atmosphere of nitrogen, was added n-butyl chloroformate (1.37 g, 1.30 mL, 10.02 mmol) and stirring was continued at room temperature overnight. The mixture was filtered and the filtrate concentrated to yield a pale yellow residue. This crude residue was purified using a Biotage Isolera automated chromatography system under normal phase conditions (silica column, gradient of 0→100% EtOAc in petrol) with detection at 254 nm to afford guanfacine n-butyl carbamate hydrochloride (830 mg, 22%), as a white solid.

Appearance: white solid; LCMS: m/z=345.85, consistent for protonated parent ion (MH⁺); ¹H NMR (DMSO-d₆): 11.27 (br, 1H, NH), 8.83 (br, 2H, NH₂⁺), 7.49 (d, J=7.7 Hz, 2H, 2×ArH), 7.35 (m, 1H, ArH), 4.06 (s, 2H, ArCH₂), 3.98 (t, J=6.5 Hz, 2H, CH₂), 1.55 (m, 2H, CH₂), 1.33 (m, 2H, CH₂), 0.89 (t, J=7.3 Hz, 3H, CH₃); Purity: >95% by HPLC, no free guanfacine by HPLC; Solubility: >10 mg/mL in DMSO, <1 mg/mL in water, >1 mg/mL in water/CH₃CN (1:1).

### Compound 7: 2-{N′-[2-(2,6-Dichloro-phenyl)-acetyl]-guanidinocarbonyloxy}-butane Hydrochloride

Trivial name: Guanfacine 2-butanol carbamate Hydrochloride

Appearance: white solid; LCMS: m/z=345.85, consistent for protonated parent ion (MH⁺); ¹H NMR (DMSO-d₆): 9.53 (br, 2H, NH₂⁺), 7.53 (d, J=7.6 Hz, 2H, 2×ArH), 7.39 (m, 1H, ArH), 4.76 (m, 1H, CH), 4.15 (s, 2H, CH₂), 1.59 (m, 2H, CH₂), 1.23 (d, J=6.3 Hz, 3H, CH₃), 0.88 (t, J=7.4 Hz, 3H, CH₃); Purity: >95% by HPLC, no free guanfacine by HPLC; Solubility: >10 mg/mL in DMSO, <1 mg/mL in water, >5 mg/mL in water/CH₃CN (1:1).

### Compound 8: 1-{N′-[2-(2,6-Dichloro-phenyl)-acetyl]-guanidinocarbonyloxy}-2-methylpropane Hydrochloride

Trivial name: Guanfacine isobutyl carbamate Hydrochloride

To a stirred suspension of guanfacine hydrochloride (2.83 g, 10.02 mmol) and 4-methylmorpholine (1.01 g, 1.10 mL, 10.02 mmol) in dry THF (60 mL), under an atmosphere of nitrogen, was added isobutyl chloroformate (1.37 g, 1.31 mL, 10.02 mmol) and stirring was continued at room temperature overnight. The mixture was filtered and the filtrate concentrated to yield a pale yellow residue. The residue was purified using a Biotage Isolera automated chromatography system under normal phase conditions (silica column, gradient of 0→100% EtOAc in petrol) with detection at 254 nm to afford guanfacine iso-butyl carbamate hydrochloride (1.10 g, 29%), as a white solid.

Appearance: white solid; LCMS: m/z=345.85, consistent for protonated parent ion (MH⁺); ¹H NMR (DMSO-d₆): 11.31 (br, 1H, NH), 8.83 (br, 2H, NH₂⁺), 7.49 (d, J=7.6 Hz, 2H, 2×ArH), 7.35 (m, 1H, ArH), 4.06 (s, 2H, ArCH₂), 3.77 (d, J=6.5 Hz, 2H, CH₂), 1.86 (m, 1H, CH), 0.89 (d, J=6.7 Hz, 6H, 2×CH₃); Purity: >95% by HPLC, no free guanfacine by HPLC; Solubility: >10 mg/mL in DMSO, <1 mg/mL in water, >1 mg/mL in water/CH₃CN (1:1).

### Compound 9: 1-{N′[2-(2,6-Dichloro-phenyl)-acetyl]guanidinocarbonyloxy}-2,2-dimethylpropane Hydrochloride

Trivial name: Guanfacine neopentyl carbamate Hydrochloride

To a stirred suspension of guanfacine hydrochloride (2.83 g, 10.02 mmol) and 4-methylmorpholine (1.01 g, 1.10 mL, 10.02 mmol) in dry THF (60 mL), under an atmosphere of nitrogen, was added neopentyl chloroformate (1.51 g, 1.49 mL, 10.02 mmol) and stirring was continued at room temperature overnight. The mixture was filtered and the filtrate concentrated to yield a pale yellow residue. This crude residue was purified using a Biotage Isolera automated chromatography system under normal phase conditions (silica column, gradient of 5→100% EtOAc in petrol) with detection at 254 nm to afford guanfacine neopentyl carbamate hydrochloride (1.33 g, 34%), as a white solid.

Appearance: white solid; LCMS: m/z=359.95, consistent for protonated parent ion (MH⁺); ¹H NMR (DMSO-d₆): 11.38 (br, 1H, NH), 8.83 (br, 2H, NH₂⁺), 7.49 (d, J=7.7 Hz, 2H, 2×ArH), 7.35 (m, 1H, ArH), 4.05 (s, 2H, ArCH₂), 3.68 (s, 2H, CH₂), 0.91 (s, 9H, 3×CH₃); Purity: >95% by HPLC, no free guanfacine by HPLC; Solubility: >10 mg/mL in DMSO, <1 mg/mL in water, >1 mg/mL in water/CH₃CN (1:1).

### Compound 10: {N′-[2-(2,6-Dichloro-phenyl)-acetyl]guanidino-carbonyloxy}toluene Hydrochloride

Trivial name: Guanfacine benzyl carbamate Hydrochloride

To a stirred suspension of guanfacine hydrochloride (1.50 g, 5.31 mmol) and 4-methylmorpholine (0.53 g, 0.58 mL, 5.31 mmol) in dry THF (30 mL), under an atmosphere of nitrogen, was added benzyl chloroformate (1.09 g, 0.91 mL, 6.37 mmol) and stirring was continued at room temperature for 5 h. The mixture was filtered and the filtrate concentrated to yield an off-white solid. The residue was purified using a Biotage Isolera automated chromatography system under reversed-phase conditions (C₁₈ column, gradient of 0→100% MeCN in 0.02% aqueous HCl) with detection at 254 nm to give, after freeze-drying, a white solid. The crude solid was triturated with diethyl ether, collected by suction filtration and dried in vacuo at 40° C. overnight to afford guanfacine benzyl carbamate hydrochloride (285 mg, 13%), as a white solid.

Appearance: white solid; LCMS: m/z=379.85, consistent for protonated parent ion (MH⁺); ¹H NMR (DMSO-d₆): 8.88 (br, 2H, NH₂⁺), 7.49 (d, J=7.8 Hz, 2H, 2×ArH), 7.40-7.28 (m, 6H, 6×ArH), 5.08 (s, 2 H, CH₂), 4.08 (s, 2H, CH₂); Purity: >99% by HPLC, no free guanfacine by HPLC; Solubility: >10 ring/mL in DMSO, <1 mg/mL in water, >5 mg/mL in CH₃CN.

### Example 3

### Alternative Synthesis for Guanfacine 2-Butanol Carbamate Hydrochloride (Compound 7)

The synthesis of guanfacine 2-butanol carbamate hydrochloride may alternatively be achieved in two reaction steps. Initially, the ‘activated carbonate’ can be prepared from 2-butanol, N,N′-disuccinimidyl carbonate (DSC) and pyridine in acetonitrile:

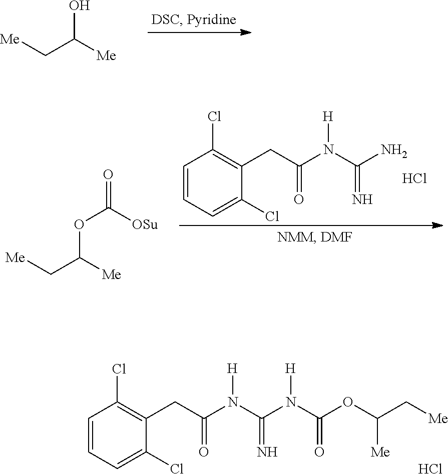

The activated carbonate can then be subsequently coupled to guanfacine hydrochloride in the presence of 4-methylmorpholine. Purification by normal phase chromatography afforded guanfacine 2-butanol carbamate. Salt formation was achieved using a solution of 2 M hydrogen chloride in diethyl ether to afford guanfacine 2-butanol carbamate hydrochloride as a white solid.

To a stirred solution of 2-butanol (0.50 g, 0.62 mL, 6.76 mmol) and pyridine (0.70 g, 0.71 mL, 8.85 mmol) in acetonitrile (40 mL) was added N,N′-disuccinimidyl carbonate (2.25 g, 8.78 mmol) in one portion, and stirring was continued at room temperature overnight. The resulting mixture was evaporated to dryness and the residue was taken up in dichloromethane (100 mL), washed with saturated aqueous sodium bicarbonate (2×100 mL) and saturated brine (50 mL), dried (MgSO4) and concentrated to afford 2-butanol-(CO.N-hydroxysuccinimide) (1.16 g, 79%), as a colourless oil that was used without further purification.

A mixture of guanfacine hydrochloride (1.81 g, 6.42 mmol) and 4-methylmorpholine (1.30 g, 1.41 mL, 12.84 mmol) in anhydrous DMF (40 mL) was stirred for 10 min at room temperature. To the stirred solution was added 2-butanol-(CO.N-hydroxysuccinimide) (1.15 g, 5.35 mmol) in anhydrous DMF (10 mL) and the mixture was stirred at room temperature overnight. The mixture was diluted with ethyl acetate (50 mL) and the solution was quenched [water containing NaCl (1.25 g per L) and AcOH (0.14 g per L)] (50 mL) with stirring for 30 min. The organic layer was separated and washed with saturated aqueous sodium bicarbonate (50 mL) and saturated brine (50 mL), dried (MgSO₄) and concentrated. The residue was purified by using a Biotage Isolera automated chromatography system under normal phase conditions (silica column, gradient of 5→40% EtOAc in petrol) with detection at 254 nm to afford crude guanfacine 2-butanol carbamate (360 mg, 19%), as a white solid.

To a stirred solution of guanfacine 2-butanol carbamate (360 mg, 1.04 mmol) in diethyl ether (14 mL) was added 2 M hydrogen chloride in diethyl ether (1.04 mL, 2.08 mmol) and stirring was continued at room temperature for 20 min. The mixture was evaporated to dryness and residual hydrogen chloride was removed azeotropically with diethyl ether (15 mL). The product was collected by suction filtration and washed diethyl ether (2×15 mL). The residue was triturated with ethyl acetate, collected by suction filtration and dried in vacuo at 40° C. overnight to afford guanfacine 2-butanol carbamate hydrochloride (195 mg, 10%), as a white solid.

Appearance: White solid. LCMS: m/z=345.85, consistent for protonated parent ion (MH⁺) 1H NMR (DMSO-d6): 11.31 (br, 1H, NH), 8.83 (br, 2H, NH₂+), 7.49 (d, J=7.6 Hz, 2H, 2×ArH), 7.35 (m, 1H, ArH), 4.06 (s, 2H, ArCH₂), 3.77 (d, J=6.5 Hz, 2H, CH₂), 1.86 (m, 1H, CH), 0.89 (d, J=6.7 Hz, 6H, 2×CH₃). Purity: >95% by HPLC. No free guanfacine by HPLC. Solubility: >10 mg/mL in DMSO, <1 mg 1 mL in water, >1 mg/mL in water/CH3CN (1:1).

### Example 4

### Preparation of Guanfacine Carbamate Prodrugs

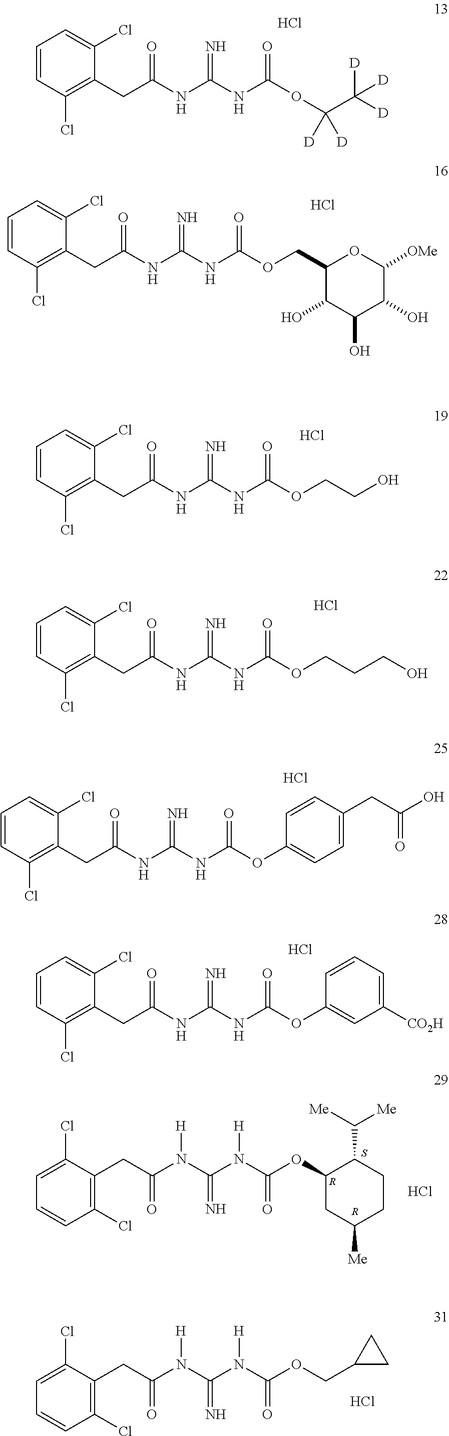

Compound 13: {N′-[2-(2,6-Dichloro-phenyl)-acetyl]-guanidinocarbonyloxy} d₅-ethane.

Trivial name: Guanfacine d₅-ethyl carbamate

A stirred solution of d₆-ethanol (1.00 g, 19.19 mmol) and pyridine (4.55 g, 4.65 mL, 57.57 mmol) in anhydrous toluene (40 mL) was cooled in an ice-bath under nitrogen and 20% phosgene in toluene (10.44 g, 11.10 mL, 21.11 mmol) was added dropwise. Stirring was continued for a further 1.5 h during which the reaction mixture was allowed to warm to room temperature. The resulting mixture was diluted with THF (100 mL), and guanfacine hydrochloride (6.51 g, 23.03 mmol) was added in one portion and stirring was continued at room temperature overnight. The red-brown mixture was filtered and the filtrate was concentrated to yield a brown residue (1.27 g). This residue was purified using a Biotage Isolera automated chromatography system under normal phase conditions (silica column, gradient of 10→100% ethyl acetate in petrol) with detection at 254 nm to afford guanfacine d₅-ethyl carbamate (0.17 g, 3%), as a yellow solid.

Appearance: yellow solid; LCMS: m/z=322.90, consistent for protonated parent ion (MH⁺); ¹H NMR (DMSO-d₆): 11.23 (br, 1H, NH), 8.83 (br, 2H, NH₂⁺), 7.49 (d, J=7.7 Hz, 2H, 2×ArH), 7.35 (m, 1H, ArH), 4.06 (s, 2H, ArCH₂); Purity: >95% by HPLC, no free guanfacine by HPLC; Solubility: >10 mg/mL in DMSO, <1 mg/mL in water.

Compound 16:

### 2-(2,6-dichlorophenyl)-N-(N-((((2R,3S,4S,5R,6S)-3,4,5-trihydroxy-6-methoxytetrahydro-2H-pyran-2-yl)meth oxy)carbonyl)carbamimidoyl)acetamide hydrochloride

Trivial name: Guanfacine-6-glucose carbamate hydrochloride

Appearance: pale yellow solid; LCMS: ES⁺ (M+H)⁺=466.06; ¹H NMR (DMSO-d₆): 3.06 (1H, t, J=10), 3.13 (1H, s), 3.17 (1H, dd, J=10, 4), 3.23 (3H, s), 3.36 (1H, m), 3.52 (1H, m), [4.10 (2H, s), 4.31 (?H, d), 4.51 (?H, d, J=4) partially obscured by broad H₂O/HCl peak], 7.35 (1H, t, J=8), 7.48 (2H, d, J=8); Purity: 89.01% area (234 nm); Solubility: water, DMSO.

Compound 19: 2-{N′-[2-(2,6-Dichloro-phenyl)-acetyl]-guanidinocarbonyloxy}ethanol

Trivial name: Guanfacine (2-hydroxyethyl) carbamate

Ethylene glycol (2.98 g, 2.68 mL, 48.0 mmol) was dissolved in anhydrous pyridine (18 mL) and tert-butyldiphenyl chlorosilane (4.40 g, 4.10 mL, 16 mmol) was slowly added to the solution at 0° C. The mixture was stirred overnight at room temperature. Pyridine was removed by vacuum distillation to give a colourless oil (4.98 g). The residue was purified using a Biotage Isolera automated chromatography system under normal phase conditions (silica column, gradient of 5→100% ethyl acetate in petrol) with detection at 254 nm to give 2-(tert-butyl-diphenyl-silanyloxy)-ethanol (3.30 g, 69%), as a white solid.

To a stirred solution of 2-(tert-butyl-diphenyl-silanyloxy)-ethanol (1.50 g, 5.00 mmol) in acetonitrile (45 mL) was added N,N′-disuccinimidyl carbonate (1.67 g, 6.50 mmol) followed by pyridine (0.51 g, 0.52 mL, 6.50 mmol) and the suspension was stirred overnight at room temperature. The resulting mixture was concentrated to dryness and the residue was taken up in dichloromethane (100 mL), washed with saturated aqueous sodium bicarbonate (3×100 mL) and saturated brine (100 mL), dried (MgSO₄) and concentrated to afford 2-(tert-butyl-diphenyl-silanyloxy)-ethanol-(CO.N-hydroxysuccinimide) (2.41 g, quantitative), as a white solid that was used without further purification.

A mixture of guanfacine hydrochloride (2.32 g, 8.20 mmol) and 4-methylmorpholine (0.83 g, 0.90 mL, 8.20 mmol) in anhydrous DMF (20 mL) was stirred for 10 min at room temperature. To this solution was added 2-(tert-butyl-diphenyl-silanyloxy)-ethanol-(CO.N-hydroxysuccininnide) (2.41 g, 5.46 mmol) in anhydrous DMF (10 mL) and the mixture was stirred at room temperature overnight. The resulting mixture was diluted with ethyl acetate (100 mL) and the solution was quenched [water containing NaCl (1.25 g per L) and AcOH (0.14 g per L)] (100 mL) with stirring for 30 min. The organic layer was separated and washed with saturated aqueous sodium bicarbonate (100 mL), water (100 mL) and saturated brine (100 mL), dried (MgSO₄) and concentrated to give a white glassy solid. This crude was purified using a Biotage Isolera automated chromatography system under normal phase conditions (silica column, gradient of 0→100% ethyl acetate in petrol) with detection at 254 nm to give guanfacine [2-(tert-butyl-diphenyl-silanyloxy)-ethyl]carbamate (2.35 g, 75%), as a white solid.

To a stirred solution of guanfacine [2-(tert-butyl-diphenyl-silanyloxy)-ethyl]carbamate (1.14 g, 2.00 mmol) in THF (20 mL) was added 1M TBAF solution in THF (4.00 mL, 4.00 mmol), and the reaction mixture was stirred at room temperature for 30 min. The resulting solution was concentrated and the residue was purified using a Biotage Isolera automated chromatography system under normal phase conditions (silica column, gradient of 0→20% methanol in dichloromethane) with detection at 254 nm to give guanfacine (2-hydroxyethyl) carbamate (0.47 g, 70%), as a white solid.

Appearance: white solid; LCMS: m/z=333.90, consistent for protonated parent ion (M Fr); ¹H NMR (DMSO-d₆): 11.23 (br, 1H, NH), 8.81 (br, 2H, 2×NH), 7.49 (d, J=7.8 Hz, 2H, 2×ArH), 7.35 (m, 1H, ArH), 4.75 (t, J=5.2 Hz, 1H, OH), 4.07 (m, 4H, ArCH₂CH₂), 3.57 (m, 2H, CH₂); Purity: >95% by HPLC, no free guanfacine by HPLC; Solubility: >10 mg/mL in DMSO, <1 mg/mL in water, >10 mg/mL in water/CH₃CN (1:1).

### Compound 22: 3-{N′-[2-(2,6-Dichloro-phenyl)-acetyl]-guanidinocarbonyloxy}propan-1-ol

Trivial name: Guanfacine (3-hydroxypropyl) carbamate

1,3-Propanediol (3.65 g, 3.47 mL, 48.0 mmol) was dissolved in anhydrous pyridine (18 mL) and tert-butyldiphenyl chlorosilane (4.40 g, 4.10 mL, 16 mmol) was slowly added to the solution at 0° C. The mixture was stirred overnight at room temperature. Pyridine was removed by vacuum distillation to give a colourless oil. The crude oil was dissolved in ethyl acetate (100 mL), washed with water (3×100 mL) and saturated brine (100 mL), dried (MgSO₄) and then concentrated to afford 3-(tert-butyl-diphenyl-silanyloxy)-propan-1-ol (5.10 g, quantitative), as a colourless oil that was used without further purification.

To a stirred solution of 3-(tert-butyl-diphenyl-silanyloxy)-propan-1-ol (1.57 g, 5.00 mmol) in acetonitrile (45 mL) was added N,N′-disuccinimidyl carbonate (1.67 g, 6.50 mmol) and then pyridine (0.51 g, 0.52 mL, 6.50 mmol) and the suspension was stirred overnight at room temperature. The resulting mixture was concentrated and the residue was taken up in dichloromethane (100 mL), washed with saturated aqueous sodium bicarbonate (3×100 mL) and saturated brine (100 mL), dried (MgSO₄) and concentrated to afford 3-(tert-butyl-diphenyl-silanyloxy)-propan-1-ol-(CO.N-hydroxysuccinimide) (2.09 g, 92%), as a white solid that was used without further purification.

A mixture of guanfacine hydrochloride (1.95 g, 6.89 mmol) and 4-methylmorpholine (0.70 g, 0.76 mL, 6.89 mmol) in anhydrous DMF (15 mL) was stirred for 10 min at room temperature. To this solution was added 3-(tert-butyl-diphenyl-silanyloxy)-propan-1-ol-(CO.N-hydroxysuccinimide) (2.09 g, 4.59 mmol) in anhydrous DMF (10 mL) and the mixture was stirred at room temperature overnight. The resulting mixture was diluted with ethyl acetate (100 mL) and the solution was quenched [water containing NaCl (1.25 g per L) and AcOH (0.14 g per L)] (100 mL) with stirring for 30 min. The organic layer was separated and washed with saturated aqueous sodium bicarbonate (100 mL), water (100 mL) and saturated brine (100 mL), dried (MgSO₄) and concentrated to give an oil. This crude material was purified using a Biotage Isolera automated chromatography system under normal phase conditions (silica column, gradient of 5→100% ethyl acetate in petrol) with detection at 254 nm to give guanfacine [3-(tert-butyl-diphenyl-silanyloxy)-propyl]carbamate (1.64 g, 61%), as a white solid.

To a stirred solution of guanfacine [3-(tert-butyl-diphenyl-silanyloxy)-propyl]carbamate (1.62 g, 2.77 mmol) in THF (25 mL) was added 1 M TBAF solution in THF (5.54 mL, 5.54 mmol), and the reaction mixture was stirred at room temperature for 30 min. The resulting solution was concentrated to dryness and the residue was purified using a Biotage Isolera automated chromatography system under normal phase conditions (silica column, gradient of 0→50% methanol in dichloromethane) with detection at 254 nm to give guanfacine (3-hydroxypropyl) carbamate (0.87 g, 90%), as a white solid.

Appearance: white solid; LCMS: m/z=347.90, consistent for protonated parent ion (MH⁺); ¹H NMR (DMSO-d₆): 11.26 (br, 1H, NH), 8.83 (br, 2H, 2×NH), 7.49 (d, J=7.8 Hz, 2H, 2×ArH), 7.35 (m, 1H, ArH), 4.51 (t, J=5.1 Hz, 1H, OH), 4.06 (m, 4H, ArCH₂CH₂), 3.47 (m, 2H, CH₂), 1.71 (m, 2H, CH₂); Purity: >95% by HPLC, no free guanfacine by HPLC; Solubility: >10 mg/mL in DMSO, <1 mg/mL in water, >10 mg/mL in water/CH₃CN (1:1).

Compound 25: 2{4-[({[2-(2,6-dichlorophenyl)acetamidol]methanimidoyl)oxy]phenyl}acetic acid Hydrochloride

Trivial name: Guanfacine phenyl acetic acid carbamate Hydrochloride

Appearance: white solid; LCMS: ES⁺ (M+H) 423.81, 425.76; ¹H NMR (DMSO-d₆): δ 9.20 (br s, 1H, NH), 9.03 (br s, 1H, NH), 7.52 (d, 2H, J=8.5, ArH), 7.37 (dd, 1H, J=8.8, 8.7, ArH), 7.28 (d, 2H, J=8.5, ArH), 7.09 (d, 2H, J=8.5, ArH), 4.14 (s, 2H, PhCH₂), 3.58 (s, 2H, CH₂CO₂H); Purity: >96.5% by HPLC; Solubility: >23 mg/mL in DMSO, <2.3 mg/mL in water.

### Compound 28: 3-[({[2-(2,6-dichlorophenyl)acetamido]methanimidoyl}carbamoyl)oxy]benzoic acid hydrochloride

Trivial name: Guanfacine meta-hydroxybenzoic acid carbamate hydrochloride

Appearance: white solid; LCMS: m/z=411, consistent for protonated parent ion (MH⁺); ¹H NMR (DMSO-d₆): 9.40 (br s, 1H), 9.0 (br s, 1H), 7.9 (d, 1H), 7.8 (s, 1H), 7.6-7.5 (m, 3H), 7.4-7.2 (m, 2H), 4.2 (s, 2H).; Purity: >96% by HPLC; Solubility: >20 mg 1 mL in DMSO, insoluble in water.

### Compound 29

### 1-(R)-{N′-[2-(2,6-Dichloro-phenyl)-acetyl]-guanidinocarbonyloxy}-2-(S)-isopropyl-5-(R)-methyl-cyclohexane Hydrochloride

Trivial name: Guanfacine-(−)-menthyl carbamate Hydrochloride

To a stirred suspension of guanfacine hydrochloride (1.50 g, 5.31 mmol) and 4-methylmorpholine (538 mg, 0.59 mL, 5.31 mmol) in dry tetrahydrofuran (30 mL), under an atmosphere of nitrogen, was added (−)-(1R)-menthyl chloroformate (1.39 g, 1.36 mL, 6.38 mmol) and stirring was continued at room temperature overnight. The mixture was filtered and the filtrate was concentrated to give a glassy solid. This crude solid was purified using a Biotage Isolera automated chromatography system under reversed-phase conditions (C₁₈ column, gradient of 0 of guanfacine hydrochloride (1.50 g, 5.31 mmol) and 4-methylmorpholine (538 mg, 0.59 mL, 5.31 mmol) in dry tetrahydrofuran (30 mL), under an atmosphere of nitrogen, was added (−)-(1R)-menthyl chloroformate (1.39 g, 1.36 mL(−)-menthyl carbamate hydrochloride (450 mg, 28%), as a white solid.

Appearance: White solid; LCMS: m/z=427.95, consistent for protonated ion (MH⁺); ¹H NMR (DMSO-d₆): 8.99 (br, 2H, NH₂^{f}), 7.50 (d, J=8.4, 2 H, 2×ArH), 7.36 (m, 1H, ArH), 4.77 (m, 1H, OCH), 4.07 (s, 2H, ArCH₂), 1.97-1.83 (m, 2H, CH and 0.5×CH₂), 1.62 (m, 2H, CH₂), 1.15 (m, 1H, 0.5×CH₂), 1.32 (m, 1H, 0.5×CH₂), 1.07-0.83 (m, 9H, 2× Isopropyl CH₃, Isopropyl CH, CH and 0.5×CH₂), 0.74 (d, J=6.9 Hz, 3H, CH₃); Purity: >95% by HPLC; No free guanfacine by HPLC; Solubility: >10 mg/mL in DMSO, <1 mg/mL in water, >5 mg in 2.5 mL CH₃CN/1 mL H₂O.

### Compound 31: {N′-[2-(2,6-Dichloro-phenyl)acetyl]-guanidino-carbonyloxy}methylcyclopropane Hydrochloride

Trivial name: Guanfacine cyclopropylmethyl carbamate Hydrochloride

To a stirred solution of cyclopropanemethanol (1.00 g, 0.90 mL, 13.87 mmol) and pyridine (1.43 g, 1.45 mL, 18.03 mmol) in acetonitrile (80 mL) was added N,N′-disuccinimidyl carbonate (4.62 g, 18.03 mmol) in one portion and the solution was heated to 40° C. for 4 h. After cooling to room temperature, the solvent was evaporated to dryness and the residue was taken up in dichloromethane (150 mL), washed with saturated aqueous sodium bicarbonate (2×150 mL) and saturated brine (100 mL), dried (MgSO₄) and concentrated to afford cyclopropanemethanol-(CO.N-hydroxysuccinimide) (2.93 g, 99%), as a colourless oil that was used without further purification.

A mixture of guanfacine hydrochloride (4.07 g, 14.40 mmol) and 4-methylmorpholine (2.91 g, 3.17 mL, 28.80 mmol) in anhydrous DMF (60 mL) was stirred for 10 min at room temperature. To the stirred solution was added cyclopropanemethanol-(CO.N-hydroxysuccinimide) (2.92 g, 13.71 mmol) in anhydrous DMF (10 mL) and the mixture was stirred at room temperature overnight. The resulting mixture was diluted with ethyl acetate (100 mL) and the solution was quenched [water containing NaCl (1.25 g per L) and AcOH (0.14 g per L)] (100 mL) with stirring for 30 min. The organic layer was separated and washed with saturated aqueous sodium bicarbonate (100 mL), water (100 mL) and saturated brine (100 mL), dried (MgSO₄) and concentrated to afford crude guanfacine cyclopropylmethyl carbamate (3.27 g), as a white solid.

A portion of the crude product (1.89 g) was purified by using a Biotage Isolera automated chromatography system under normal phase conditions (silica column, gradient of 5→40% EtOAc in petrol) with detection at 254 nm to afford guanfacine cyclopropylmethyl carbamate (0.89 g, 33%), as a white solid.

To a stirred solution of guanfacine cyclopropylmethyl carbamate (0.88 g, 2.56 mmol) in diethyl ether (34 mL) was added 2 M hydrogen chloride in diethyl ether (2.56 mL, 5.12 mmol) and stirring was continued at room temperature for 20 min. The mixture was evaporated to dryness and residual hydrogen chloride was removed azeotropically with diethyl ether (2×25 mL). The product was collected by suction filtration and dried in vacuo at 40° C. overnight to afford guanfacine cyclopropylmethyl carbamate hydrochloride (95 mg, 97%) as a white solid.

Appearance: White solid; LCMS: m/z=343.95, Consistent for protonated parent ion (MH⁺); ¹H NMR (DMSO-d₆): 9.55 (br, 2H, NH₂⁺), 7.53 (d, J=7.7 Hz, 2H, 2×ArH), 7.39 (m, 1H, ArH), 4.17 (s, 2H, CH₂), 4.02 (d, J=7.3 Hz, 2H, CH₂), 1.15 (m, 1H, CH), 0.55 (m, 2H, 2×0.5 CH₂), 0.32 (m, 2H, 2×0.5 CH₂); Purity: >95° A, by HPLC; No free guanfacine by HPLC; Solubility: >10 mg/mL in DMSO; <1 mg/mL in water; >5 mg/mL in CH₃CN:water (1:1).

### Example 5

### Preparation of Guanfacine d₅-Ethyl Carbamate (Compound 13)

Compound 11 is activated by reacting with N,N′-disuccinimidyl carbonate (DSC) in the presence of a base, pyridine. The activated carbonate is coupled with guanfacine in the presence of NMM, followed by acidification to give the product, compound 13, as HCl salt. The synthetic route is shown below in Scheme 2.

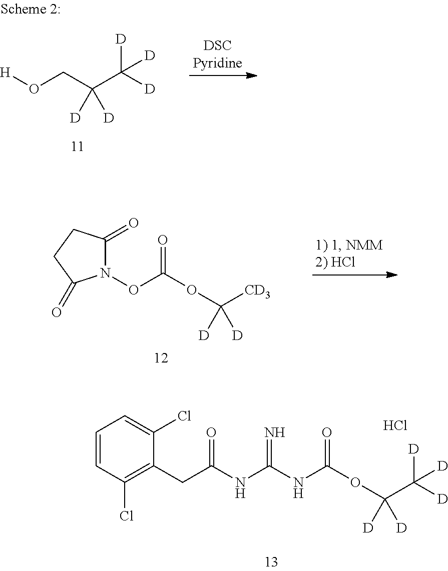

### Example 6

### Preparation of Guanfacine-6-Glucose Carbamate Hydrochloride (Compound 16)

Compound 14 is activated by reacting with N,N′-disuccinimidyl carbonate (DSC) in the presence of a base, pyridine. The activated carbonate is coupled with guanfacine in the presence of NMM to give the coupled intermediate. The intermediate is deprotected by palladium-catalyzed hydrogenation, followed acidification to give the product, compound 16, as HCl salt. The synthetic route is shown below in Scheme 3.

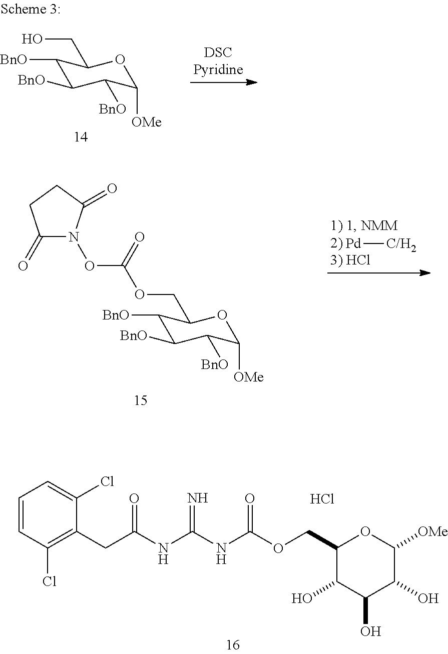

### Example 7

### Preparation of Guanfacine (2-Hydroxyethyl) Carbamate (Compound 19)

Compound 17 is activated by reacting with N,N′-disuccinimidyl carbonate (DSC) in the presence of a base, pyridine. The activated carbonate is coupled with guanfacine in the presence of NMM to give the coupled intermediate. The intermediate is deprotected by palladium-catalyzed hydrogenation, followed acidification to give the product, compound 19, as HCl salt. The synthetic route is shown below in Scheme 4.

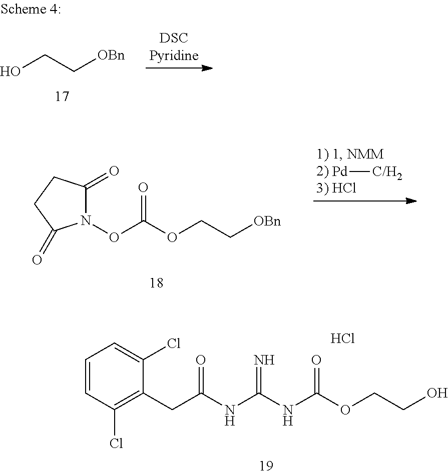

### Example 8

### Preparation of Guanfacine (2-Hydroxyethyl) Carbamate (Compound 22)

Compound 20 is activated by reacting with N,N′-disuccinimidyl carbonate (DSC) in the presence of a base, pyridine. The activated carbonate is coupled with guanfacine in the presence of NMM to give the coupled intermediate. The intermediate is deprotected by palladium-catalyzed hydrogenation, followed acidification to give the product, compound 22, as HCl salt. The synthetic route is shown below in Scheme 5.

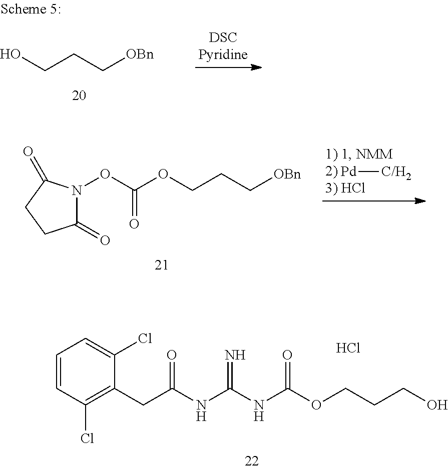

### Example 9

### Preparation of Guanfacine Phenyl Acetic Acid Carbamate Hydrochloride (Compound 25)

Compound 23 is activated by reacting with N,N′-disuccinimidyl carbonate (DSC) in the presence of a base, pyridine. The activated carbonate is coupled with guanfacine in the presence of NMM to give the coupled intermediate. The intermediate is deprotected by palladium-catalyzed hydrogenation, followed acidification to give the product, compound 25, as HCl salt. The synthetic route is shown below in Scheme 6.

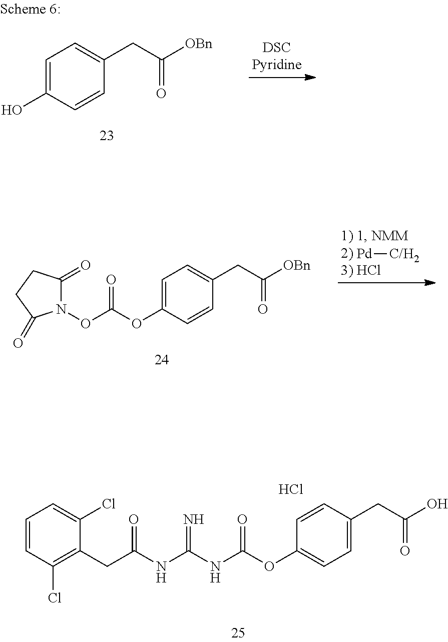

### Example 10

### Preparation of Guanfacine Meta-Hydroxybenzoic Acid Carbamate Hydrochloride (Compound 28)

Compound 26 was activated by reacting with N,N′-disuccinimidyl carbonate (DSC) in the presence of a base, pyridine. The activated carbonate was coupled with guanfacine in the presence of NMM to give the coupled intermediate. The intermediate was deprotected by palladium-catalyzed hydrogenation, followed acidification to give the product, compound 28, as HCl salt. The synthetic route is shown below in Scheme 7.

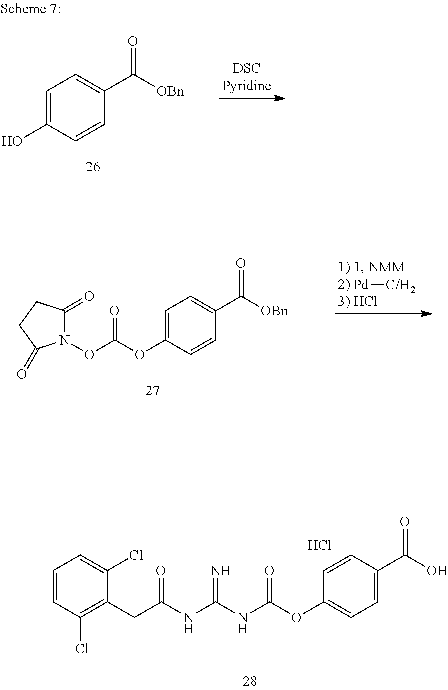

### Example 11

### Preparation of Guanfacine-(S)-histidinyl carbamate Tri-trifluoroacetate

The synthesis of guanfacine-(S)-histidinyl carbamate tri-trifluoroacetate was achieved in four distinct steps. Initially, S-histidinol was protected by treatment with di-tert-butyl dicarbonate to give N,N′-di-Boc-histidinol in good yield. The protected histidinol was converted to the ‘activated carbonate’ with N,N′-disuccinimidyl carbonate followed by coupling of this ‘activated carbonate’ to guanfacine to give N,N′-di-Boc-(S)-histidinyl-guanfacine carbamate:

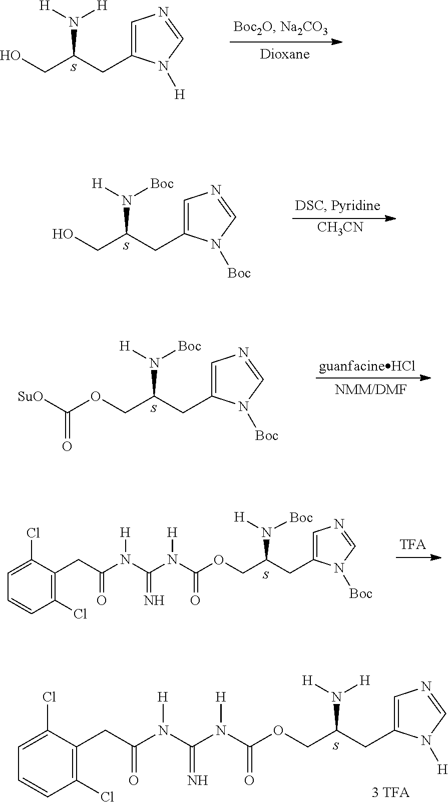

Removal of the Boc groups was achieved by treatment with trifluoroacetic acid to give guanfacine-(S)-histidinyl carbamate tri-trifluoroacetate as a white solid, following purification by reversed-phase chromatography.

To a stirred solution of L-histidinol dihydrochloride (1.00 g, 4.67 mmol) in dioxane (29 mL) in an ice-bath was added a solution of sodium carbonate (4.63 g, 43.71 mmol) in water (14 mL) followed by di-tert-butyl dicarbonate (2.24 g, 10.27 mmol) and the mixture was stirred at room temperature for 3 h. The reaction mixture was neutralised to pH 7-8 by potassium di-hydrogen phosphate, diluted with water (50 mL) and extracted with ethyl acetate (2×50 mL). The organics were combined, dried (MgSO₄) and concentrated to give a colourless oil. The crude material was purified using a Biotage Isolera automated chromatography system under normal phase conditions (silica column, gradient of 0->35% methanol in dichloromethane) with detection at 254 nm to afford N,N′-di-Boc-(S)-histidinol (1.31 g, 82%), as a white solid.

To a stirred solution of N,N′-di-Boc-(S)-histidinol (0.60 g, 1.76 mmol) in anhydrous acetonitrile (15 mL) under nitrogen was added N,N′-disuccinimidyl carbonate (0.59 g, 2.29 mmol) followed by anhydrous pyridine (0.18 g, 0.18 mL, 2.29 mmol) and the suspension was stirred overnight at room temperature. The resulting solution was concentrated and re-dissolved in dichloromethane (50 mL), washed with saturated aqueous sodium bicarbonate (3×50 mL), water (50 mL) and saturated brine (50 mL), dried (MgSO₄) and concentrated to give N,N′-di-Boc-(S)-histidinyl-CO.N-hydroxysuccinimide (0.86 g, quantitative), as an oil.

To a stirred solution of guanfacine hydrochloride (0.76 g, 2.68 mmol) and 4-methylmorpholine (0.27 g, 0.29 mL, 2.68 mmol) in dry DMF (10 mL) was added N,N′-di-Boc-(S)-histidinyl-CO.N-hydroxysuccinimide (0.86 g, 1.78 mmol) and stirring was continued at room temperature overnight. Ethyl acetate (50 mL) was added and the mixture was quenched [water containing NaCl (1.25 g per L) and AcOH (0.14 g per L] (50 mL) with stirring for 30 min. The organic layer was separated and washed with 8% aqueous sodium bicarbonate (50 mL), water (50 mL) and saturated brine (50 mL), dried (MgSO₄) and concentrated. The residue was purified using a Biotage Isolera automated chromatography system under normal phase conditions (silica column, gradient of 0→25% methanol in dichloromethane) with detection at 254 nm to afford N,N′-di-Boc-(S)-histidinyl-guanfacine carbamate (0.91 g, 84%), as a white solid.

N,N′-di-Boc-(S)-histidinyl-guanfacine carbamate (0.53 g, 0.87 mmol) in trifluoroacetic acid (20 mL) was stirred at room temperature for 1 h. The mixture was evaporated to dryness and residual trifluoroacetic acid was removed azeotropically with chloroform (4×20 mL). The residue was purified using a Biotage Isolera automated chromatography system under reversed-phase conditions (C₁₈ column, gradient of 0→100% MeCN in 0.5% aqueous TFA) with detection at 254 nm to afford, after freeze-drying, guanfacine-(S)-histidinyl carbamate tri-trifluoroacetate (0.36 g, 55%), as a white solid.

Appearance: White solid; LCMS: m/z=412.95, consistent for protonated parent ion. (MH⁺); ¹H NMR (DMSO-d₆): 14.47 (br, 2H, NH₂⁺), 9.06 (m, 3H, CH and NH₂⁺), 8.29 (br, 3H, NH₃′), 7.51 (m, 3H, 2×ArH and CH), 7.37 (m, 1H, ArH), 4.11 (m, 4H, CH₂ and ArCH₂), 3.77 (br, 1H, CH), 3.03 (m, 2H, CH₂); Purity: >95% by HPLC, No free guanfacine by HPLC; Solubility: >20 mg/mL in DMSO, >20 mg/mL in water.

### Example 12

### Preparation of Guanfacine Ethanethiol Carbamate Hydrochloride (Compound 30)

The synthesis of guanfacine ethanethiol carbamate hydrochloride was achieved in a single reaction step:

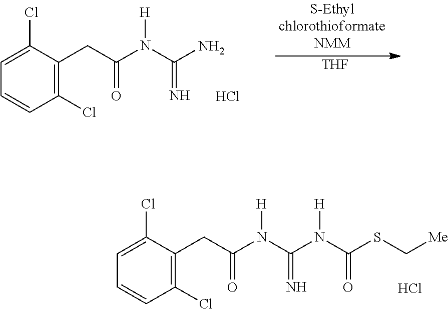

Guanfacine hydrochloride was reacted with 5-ethyl chlorothioformate in the presence of N-methylmorpholine to give the required guanfacine ethanethiol carbamate hydrochloride as a white solid.

To a stirred suspension of guanfacine hydrochloride (1.50 g, 5.31 mmol) and 4-methylmorpholine (538 mg, 0.58 mL, 5.31 mmol) in dry tetrahydrofuran (30 mL), under an atmosphere of nitrogen, was added S-ethyl chlorothioformate (795 mg, 0.67 mL, 6.38 mmol) and stirring was continued at room temperature overnight. The residue was purified by medium-pressure chromatography on silica eluting with a gradient of 20→50% ethyl acetate in petrol and dried in vacuo at 50° C. for 4 h to afford guanfacine ethanthiol carbamate hydrochloride (523 mg, 27%), as a white solid.

R_{f} 0.51 [20% ethyl acetate-80% petrol].

Appearance: White solid; LCMS: m/z=333.80, consistent for protonated parent ion (MH); ¹H NMR (DMSO-d₆): 11.06 (br, 1H, NH), 8.89 (br, 2H, NH₂⁺), 7.51 (d, J=8.4 Hz, 2H, 2×ArH), 7.36 (m, 1H, ArH), 4.09 (s, 2H, ArCH₂), 2.72 (q, J=7.2 Hz, 2H, CH₂), 0.89 (t, J=7.2 Hz, 3H, CH₃); Purity: >99% by HPLC; No free guanfacine by HPLC; >10 mg/mL in DMSO; <1 mg/mL in water; >5 mg/mL in water/CH₃CN (1:2).

### Example 13

### Preparation of Guanfacine Tert-Butyl Carbamate Hydrochloride

The synthesis of guanfacine tert-butyl carbamate hydrochloride was achieved in two reaction steps:

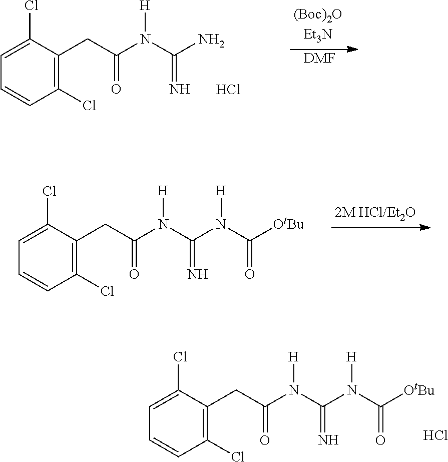

Guanfacine hydrochloride was reacted with di-tert-butyl dicarbonate in the presence of triethylamine to give guanfacine tert-butyl carbamate as a white solid after purification by normal phase chromatography. The free base was treated with a solution of 2 M hydrogen chloride in diethyl ether to afford guanfacine tert-butyl carbamate hydrochloride as a white solid.

To a stirred solution of guanfacine hydrochloride (1.00 g, 3.54 mmol) in DMF (5 mL) was added a solution of di-tert-butyl dicarbonate (1.55 g, 7.09 mmol) in DMF (5 mL) followed by triethylamine (1 mL) and the mixture was stirred overnight at room temperature. The reaction was quenched with water (50 mL) and the aqueous extracted with ethyl acetate (3×50 mL). The organics were combined and washed with water (5×100 mL), saturated brine (100 mL), dried (MgSO₄) and concentrated to give a white solid. The residue was purified using a Biotage Isolera automated chromatography system under normal phase conditions (silica column, gradient of 0→38% ethyl acetate in petrol) with detection at 254 nm to afford guanfacine tert-butyl carbamate (0.96 g, 78%), as a white solid. R_{f} 0.46 [20% ethyl acetate-80% petrol].

To a stirred suspension of guanfacine tert-butyl carbamate (0.30 g, 0.88 mmol) in diethyl ether (2 mL) was added a solution of 2 M hydrogen chloride in diethyl ether (0.44 mL, 0.88 mmol). The resulting suspension was stirred for 1 min and the solid was collected by suction filtration and dried in vacuo at 35° C. overnight to afford guanfacine Cert-butyl carbamate hydrochloride (0.32 g, 95%)/0), as a white solid.

Appearance: White solid; LCMS: m/z=345.90, consistent for protonated ion (MK); ¹H NMR (DMSO-d₆): 9.45 (br, 2H, NH₂⁺), 7.52 (d, J=8.1 Hz, 2H, 2×ArH), 7.38 (m, 1H, ArH), 4.14 (s, 2H, ArCH₂), 1.47 (s, 9H, tert-butyl); Purity: >99% by HPLC; 0.3% guanfacine by HPLC; Solubility: >10 mg/mL in DMSO, <1 mg/mL in water, >5 mg in 2 mL CH₃CN/1 mL H₂O.

### Example 14

### Stability Studies on Various Guanfacine Prodrugs

### Guanfacine Benzyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 90.7 | 0.0 | 0.0 | 100 | 100.4 | 0.0 | 0.0 | 100 | 94.0 | 0.0 | 0.0 | 100 | 98.6 | 0.0 | 0.0 |
| 100 | 90.3 | 0.0 | 0.0 | 100 | 100.5 | 0.0 | 0.0 | 100 | 95.3 | 0.0 | 0.0 | 100 | 99.0 | 0.0 | 0.0 |

### Guanfacine N-Butyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 94.0 | 0.0 | 0.0 | 100 | 94.6 | 0.0 | 0.0 | 100 | 90.9 | 0.0 | 0.0 | 100 | 90.7 | 0.0 | 0.0 |
| 100 | 94.0 | 0.0 | 0.0 | 100 | 94.9 | 0.0 | 0.0 | 100 | 90.7 | 0.0 | 0.0 | 100 | 91.0 | 0.0 | 0.0 |

### Guanfacine Isobutyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 95.1 | 0.0 | 0.0 | 100 | 95.2 | 0.0 | 0.0 | 100 | 98.2 | 0.0 | 0.0 | 100 | 95.3 | 0.0 | 0.0 |
| 100 | 95.0 | 0.0 | 0.0 | 100 | 95.3 | 0.0 | 0.0 | 100 | 97.9 | 0.0 | 0.0 | 100 | 96.7 | 0.0 | 0.0 |

### Guanfacine-(−)-Menthyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 94.6 | 0.0 | 0.0 | 100 | 98.3 | 0.0 | 0.0 | 100 | 97.9 | 0.0 | 0.0 | 100 | 94.7 | 0.0 | 0.0 |
| 100 | 95.5 | 0.0 | 0.0 | 100 | 99.6 | 0.0 | 0.0 | 100 | 97.3 | 0.0 | 0.0 | 100 | 96.9 | 0.0 | 0.0 |

### Guanfacine 2-Butanol Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 95.9 | 0.0 | 0.0 | 100 | 98.7 | 0.0 | 0.0 | 100 | 93.4 | 0.0 | 0.0 | 100 | 93.4 | 0.0 | 0.0 |
| 100 | 95.9 | 0.0 | 0.0 | 100 | 98.9 | 0.0 | 0.0 | 100 | 93.4 | 0.0 | 0.0 | 100 | 93.6 | 0.0 | 0.0 |

### Guanfacine Neopentyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 94.7 | 0.0 | 0.0 | 100 | 96.1 | 0.0 | 0.0 | 100 | 98.3 | 0.0 | 0.0 | 100 | 99.2 | 0.0 | 0.0 |
| 100 | 93.9 | 0.0 | 0.0 | 100 | 96.8 | 0.0 | 0.0 | 100 | 97.9 | 0.0 | 0.0 | 100 | 98.5 | 0.0 | 0.0 |

### Guanfacine-(S)-Histidinyl Carbamate Tri-Trifluoroacetate

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 99.5 | 84.7 | 0.5 | 1.7 | 99.6 | 98.1 | 0.4 | 1.2 | 98.4 | 0.0 | 1.6 | 87.1 | 99.2 | 0.0 | 0.8 | 85.4 |
| 99.4 | 83.3 | 0.6 | 1.8 | 99.5 | 97.8 | 0.5 | 1.3 | 98.6 | 0.0 | 1.4 | 88.1 | 98.1 | 0.0 | 1.8 | 85.8 |

### Guanfacine N-Propyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 94.4 | 0.0 | 0.0 | 100 | 94.0 | 0.0 | 0.0 | 100 | 95.8 | 0.0 | 0.0 | 100 | 96.3 | 0.0 | 0.0 |
| 100 | 94.6 | 0.0 | 0.0 | 100 | 94.4 | 0.0 | 0.0 | 100 | 96.4 | 0.0 | 0.0 | 100 | 96.0 | 0.0 | 0.0 |

### Guanfacine Ethanethiol Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 81.0 | 0.0 | 10.1 | 100 | 85.6 | 0.0 | 0.0 | 100 | 93.0 | 0.0 | 0.0 | 100 | 94.5 | 0.0 | 0.0 |
| 100 | 80.4 | 0.0 | 11.0 | 100 | 84.8 | 0.0 | 0.0 | 100 | 92.5 | 0.0 | 0.0 | 100 | 95.2 | 0.0 | 0.0 |

### Guanfacine Tert-Butyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 91.2 | 0.0 | 8.8 | 99.7 | 100 | 34.5 | 0.0 | 66.6 | 100 | 83.6 | 0.0 | 14.2 | 100 | 84.2 | 0.0 | 11.0 |
| 90.5 | 0.0 | 9.5 | 100 | 100 | 34.5 | 0.0 | 65.5 | 100 | 84.9 | 0.0 | 15.2 | 100 | 82.7 | 0.0 | 11.2 |

### Guanfacine (2-Hydroxyethyl) Carbamate

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 91.4 | 0.0 | 0.5 | 100 | 98.0 | 0.0 | 0.4 | 100 | 87.4 | 0.0 | 9.8 | 100 | 72.5 | 0.0 | 22.6 |
| 100 | 91.5 | 0.0 | 0.5 | 100 | 98.0 | 0.0 | 0.4 | 100 | 87.5 | 0.0 | 9.8 | 100 | 73.0 | 0.0 | 22.3 |

### Guanfacine D₅-Ethyl Carbamate

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 97.6 | 0.0 | 0.0 | 100 | 98.7 | 0.0 | 0.0 | 100 | 99.4 | 0.0 | 0.0 | 100 | 99.4 | 0.0 | 0.0 |
| 100 | 96.6 | 0.0 | 0.0 | 100 | 98.3 | 0.0 | 0.0 | 100 | 99.2 | 0.0 | 0.0 | 100 | 99.2 | 0.0 | 0.0 |

### Guanfacine (3-Hydroxypropyl) Carbamate

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 94.8 | 0.0 | 0.0 | 100 | 98.1 | 0.0 | 0.0 | 100 | 99.2 | 0.0 | 0.0 | 100 | 98.2 | 0.0 | 0.0 |
| 100 | 94.9 | 0.0 | 0.0 | 100 | 98.3 | 0.0 | 0.0 | 100 | 99.7 | 0.0 | 0.0 | 100 | 98.2 | 0.0 | 0.0 |

### Guanfacine Cyclopropylmethyl Carbamate Hydrochloride

| pH 1.2, 37° C. | | | | pH 3.0, 20° C. | | | | pH 6.8, 37° C. | | | | pH 7.4, 37° C. | | | |
|---|---|---|---|---|---|---|---|---|---|---|---|---|---|---|---|
| 0.1M HCl/NaCl buffer | | | | 0.1M citrate buffer | | | | 0.1M phosphate buffer | | | | 0.1M phosphate buffer | | | |
| Pro-drug | | Active drug | | Pro-drug | | Active drug | | Pro-drug | | Active drug | | Pro-drug | | Active drug | |
| 0 | 1 h | 0 | 1 h | 0 | 2 h | 0 | 2 h | 0 | 2 h | 0 | 2 h | 0 | 2 h | 0 | 2 h |
| 100 | 92.9 | 0.0 | 0.6 | 100 | 98.3 | 0.0 | 0.0 | 100 | 97.6 | 0.0 | 0.0 | 100 | 99.6 | 0.0 | 0.0 |
| 100 | 92.7 | 0.0 | 0.6 | 100 | 98.5 | 0.0 | 0.0 | 100 | 97.4 | 0.0 | 0.0 | 100 | 99.5 | 0.0 | 0.0 |

### Example 15

### Comparative In Vivo Screening Study of Guanfacine Prodrugs in the Monkey

Test substances e.g. guanfacine (0.5 mg/kg free base) and various guanfacine prodrugs at equimolar doses to that given of the parent drug were administered by oral gavage to groups of two monkeys using a multi-way crossover design.

Blood samples were taken on 4 sampling occasions at various times up to 6 h after administration and submitted to analysis for the parent drug and prodrug using a qualified LC-MS-MS assay. The relative Cₘₐₓ for guanfacine was calculated by comparison with guanfacine-dosed animals. The results are given in the table below.

| | | Rel |
|---|---|---|
| Compound | Trivial name | Cₘₐₓ |
| 3 | Guanfacine ethyl carbamate | 81 |
| 4 | Guanfacine n-propyl carbamate | 66 |
| 5 | Guanfacine isopropyl carbamate | 111 |
| 6 | Guanfacine n-butyl carbamate | 112 |
| 7 | Guanfacine 2-butanol carbamate | 61 |
| 8 | Guanfacine isobutyl carbamate | 56 |
| 9 | Guanfacine neopentyl carbamate | 42 |
| 10 | Guanfacine benzyl carbamate | 32 |
| 13 | Guanfacine d₅-ethyl carbamate | 87 |
| 16 | Guanfacine-6-glucose carbamate | 43 |
| 19 | Guanfacine (2-hydroxyethyl) carbamate | 53 |
| 22 | Guanfacine (3-hydroxypropyl) carbamate | 106 |
| 25 | Guanfacine phenyl acetic acid carbamate | 53 |
| 28 | Guanfacine meta-hydroxybenzoic acid carbamate | 53 |
| | Guanfacine glycolic acid carbamate | 10 |
| 29 | Guanfacine menthyl carbamate | 0 |
| 31 | Guanfacine cyclopropylmethyl carbamate | 82 |

### Example 16

### Comparative Bioavailability Study of Guanfacine in Monkeys Given Guanfacine or Guanfacine Prodrug

In order to characterize the pharmacokinetics of selected guanfacine conjugates, test substances e.g. guanfacine and guanfacine prodrugs were administered at equimolar doses to monkeys (0.5 mg/kg) and rats (1 mg/kg).

Blood samples were taken at various times after administration and submitted to analysis for the parent drug and prodrug using a qualified LC-MS-MS assay. The following pharmacokinetic parameters derived from the plasma analytical data were determined using Win Nonlin;

Cmax Maximum measured concentration
Frel % Relative oral bioavailability of Guanfacine

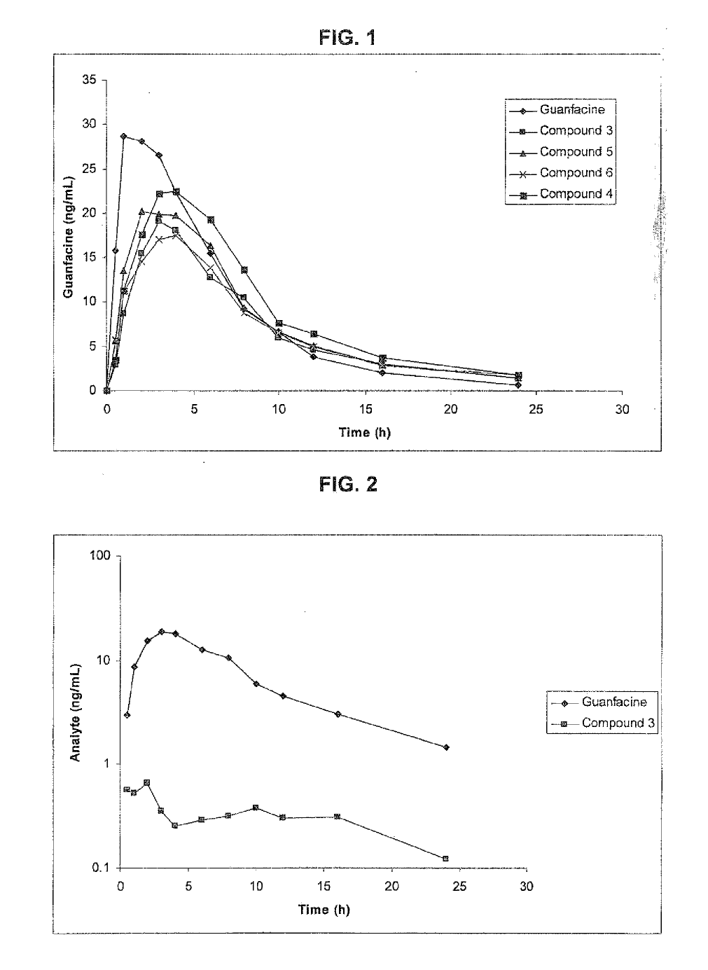

The results are given in Table 2 below and FIGS. 1 and 2 (compounds 3, 4, 5 and 6).

TABLE 2
Guanfacine pharmacokinetic parameters following administration of
guanfacine prodrugs to monkeys

| | Fᵣₑₗ | Cmax | Prodrug |
|---|---|---|---|
| Compound | % | (ng/mL) | Cmax |
| 3 | 87 | 20.3 | 0.93 |
| 4 | 113 | 24 | BLQ |
| 5 | 91 | 22.1 | BLQ |
| 6 | 89 | 18.5 | BLQ |
| 19 | 80 | 24.6 | 60.8 |
| 22 | 112 | 29.5 | 3.49 |
| 30 | NC | 5.14 | BLQ |

NC = not calculated

Administration of compounds 3,4,5 and 6 resulted in a high relative guanfacine bioabailability (>87%) with a blunted and delayed Cₘₐₓ value. (FIG. 1). Systemic levels of prodrugs were low or below the limit of quantification but detectable up to 24 hours after the administration for compound 3. (FIG. 2). The pharmacokinetic profile suggests a slow but near complete absorption of the prodrug with rapid conversion to guanfacine.

### Example 17

### The Pharmacokinetics of Guanfacine and Prodrugs in Rats in Hepatic Portal and Tail Veins Following Oral Administration of Prodrug

The absorption of intact prodrug and conversion of prodrug to guanfacine after absorption is important if any local effects of the active compound on alpha 2 adrenoceptors in the gastrointestinal tract are to be minimised. The collection of blood from the hepatic portal vein following oral administration allows the analysis of absorbed prodrug and active drug levels prior to first pass metabolism in the liver. Systemic levels can be measured by sampling of blood from the tail vein.

Methodology

Rats were surgically prepared under isofluorane anaesthesia by attaching a silicon catheter to the portal vein then exteriorising it at the nape of the neck with a blood collection port attached.

Oral doses of compound 3 were administered by gavage as a single bolus dose at a dose volume of 10 mL/kg.

At each sampling time serial point blood samples (approximately 0.2 mL) were taken simultaneously from the lateral tail vein cannula and the hepatic portal cannula. After collection of the final blood sample each animal was killed by cervical dislocation. Blood samples were collected at 15, 30 minutes and 1, 2, 4, 8 and 24 hours post dose.

Pharmacokinetic parameters in portal and systemic plasma were derived by non-compartmental analysis (linear/logarithmic trapezoidal) using WinNonlin (Version 4.1) software.

Results

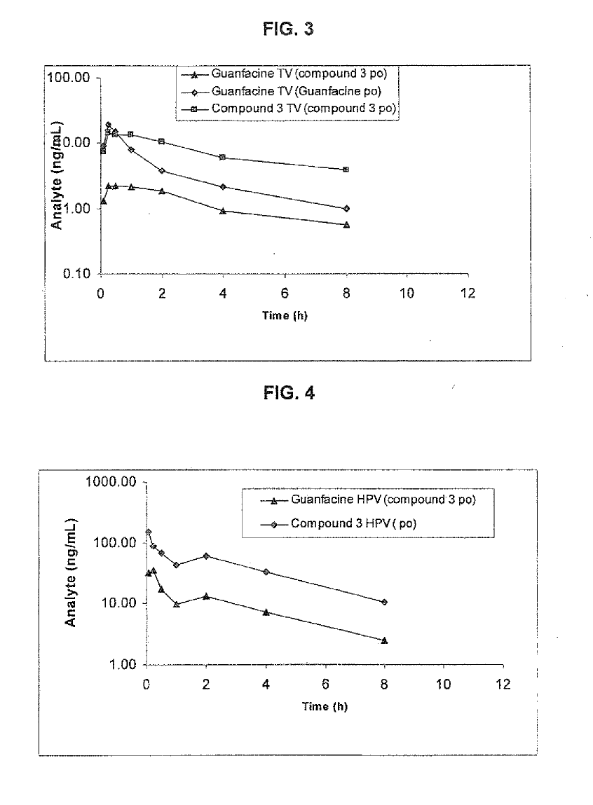

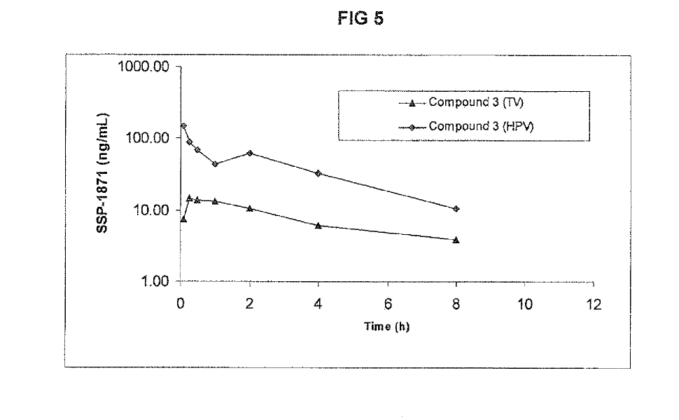

The results are presented in table 3 and in FIGS. 3, 4 and 5.

TABLE 3
Compound 3; Pharmacokinetic parameters in hepatic portal vein and tail vein following
oral administration to rat at 1 mg/kg guanfacine free base equivalents

| | Hepatic portal vein | | | | Tail vein | | | |
|---|---|---|---|---|---|---|---|---|
| | Prodrug | | Guanfacine | | Prodrug | | Guanfacine | |
| | Cmax | AUC | Cmax | AUC | Cmax | AUC | Cmax | AUC |
| Compound | (ng/mL) | (ng · h/mL) | (ng/mL) | (ng · h/mL) | (ng/mL) | (ng · h/mL) | (ng/mL) | (ng · h/mL) |
| 3 | 149 | 397 | 44.3 | 72.9 | 16.4 | 88.6 | 2.84 | 11.7 |

The substantial presence of the prodrug in the hepatic portal circulation relative to the concentration in the systemic circulation demonstrated the absorption of the prodrug prior to absorption across the intestine and confirmed adequate stability in the intestinal lumen. This suggests a lack of extensive degradation of compound 3 prior to absorption and a reduction in the potential to elicit a direct pharmacological effect in the gut lumen.

### Example 18

### In Vitro Assessment of the Effects of Guanfacine and Guanfacine Prodrug on a-2 Adrenoceptor Binding

The target receptor for guanfacine is the human alpha adrenergic 2A receptor subtype in the central nervous system. The activation of this receptor is responsible for its intended therapeutic effect. However, it is possible that local activation of this receptor which is also present in the gut contributes to adverse gastrointestinal effects (constipation) associated with guanfacine. The receptor binding of the prodrugs was investigated to confirm that the prodrug molecules had been largely inactivated.

Methods

The binding assay methodology employed in this study followed that described by Langin et al. (Eur. J. Pharmacol. 167:95-104, 1989) and used human recombinant CHO cells expressing a-2 adrenceptors. The competitive binding ligand was [3H] RX821002 (1 nM) which has a high affinity for the alpha-2A subtype.

Results

The results are set forth in Table 4. Guanfacine in non-prodrug form showed considerable potency as a competitive binding agent at the a-2A adrenoceptor displaying an Ki of 32 nM, The prodrug tested in the assay was a less potent binding agent to the receptor. The Ki value of prodrug was 300-fold greater than that obtained with guanfacine. Thus, the prodrug described herein would have little or no effect on intestinal a-2A adrenoceptors and hence potentially have a diminished ability to induce constipation through direct actions on gut motility, compared to guanfacine in non-prodrug form.

TABLE 4
Binding of guanfacine and prodrug at a-2A adrenoceptor

| Compound | Ki |
|---|---|
| Guanfacine | 32 nM |
| 3 | >10 μM |

### Example 19

### In Vivo Effects of Guanfacine and its Prodrugs on Gut Motility in Rat

The effect of a drug on gut motility can be studied by means of the charcoal propulsion test. Drugs known to cause constipation such as morphine and guanfacine significantly delay the transit of a charcoal meal in the rat. The effects of guanfacine in non-prodrug form and its prodrug on GI motility were assessed in rats fasted overnight prior to the test.

The method used was based on that described by Takemori et al. (J. Pharmacol. Exp. Ther. 169:39, 1969). Test treatments were administered orally 60 minutes prior to an oral dose of a 10% suspension of charcoal in 2.5% gum Arabic (2 ml/kg). Twenty minutes after dosing with charcoal, the rats were sacrificed and the entire gastrointestinal tract was removed quickly and carefully. The distance that the charcoal meal had traveled toward the caecum was measured and expressed as a percentage of the total gut length. The results are described in Table 5.

Orally administered guanfacine in non-prodrug form at a dose of 0.1 mg base/kg had significant effects on gut motility with about 41% reduction in the distance traveled by the charcoal plug within 20 minutes, compared to that of the control group (treated with the vehicle). The prodrug was considerably less potent than guanfacine in the inhibition of GIT transit in the rat (table 5), although systemic plasma guanfacine levels were similar after administration of either compound 3 or guanfacine. (table 6).

TABLE 5
Effects of guanfacine or guanfacine prodrug on gastrointestinal transit
of a charcoal meal in the rat

| | % inhibition of | | |
|---|---|---|---|
| | rat GIT transit at | | |
| | dose (guanfacine | | |
| | free base | | |
| | equivalents) | | |
| | mg/kg | | |
| Compounds | 00.1 | 00.3 | 100 |
| Guanfacine | 41 | 56 | — |
| 3 | 15 | 56 | 57 |

TABLE 6
Systemic plasma levels of guanfacine in rats after oral administration
of compound 3 or guanfacine at equimolar doses

| | Dose (guanfacine free base equivalents) | | | | | |
|---|---|---|---|---|---|---|
| | 0.1 mg/kg | | 0.3 mg/kg | | 1 mg/kg | |
| | Systemic plasma concentration of guanfacine | | | | | |
| Compounds | (ng/mL) at 60 and 80 minutes after dosing | | | | | |
| administered | 60 min | 80 min | 60 min | 80 min | 60 min | 80 min |
| Guanfacine | 0.62 | 0.40 | 0.36 | 1.00 | NM | NM |
| 3 | 0.51 | 0.27 | 1.21 | 0.87 | 2.45 | 2.17 |

Without being by bound by any theory, the lack of effects on gut motility by the prodrug is attributed in part to the reduced or minimally available active drug (guanfacine) within the gut lumen to interact locally with a-2A adrenoceptors.

### Example 20

### In Vivo Effects of Prodrug in Anxiety Models in Rodents

The effects of compound 19 was evaluated in three rodent models of efficacy and compound 19 was shown to be efficacious in the Elevated Plus-Maze Test in the rat.

The method, which detects anxiolytic activity, follows that described by Handley and Mithani (Naunyn. Schmied. Arch. Pharmacol., 327, 1-5, 1984). Rodents avoid open spaces (the open arms of an elevated plus-maze). The percentage of time spent on the open arms and the number of entries in the open arms are considered to be the best measures of anxiety (File, S. E. 1991. Animal models of anxiety. In Biological Psychiatry (G. Racagni, N. Brunello, and T. Fukuda, eds.) pp. 596-599. Elsevier, New York.).

Anxiolytics increase exploratory activity in the open arms, as indicated by increased time spent on the open arms and/or by increased % open-arm entries.

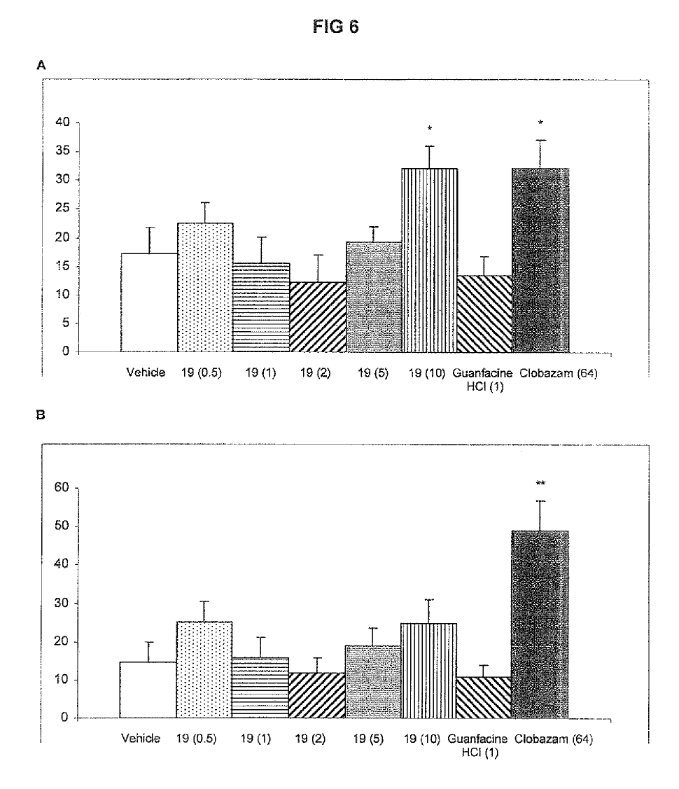

At 10 mg/kg, compound 19 significantly increased the percent of entries and had a similar tendency on the time spent in the open arms (+87%, p<0.05 and +69%, NS, respectively) (FIG. 6).

### Example 21

### Comparative Bioavailability of Guanfacine in a Microdose Study in Healthy Adult Male and Female Subjects Given Guanfacine or Guanfacine Prodrug (Compound 3)

This study determined the extent to which compound 3 is orally absorbed and cleaved systemically to release guanfacine.

Compound 3 and guanfacine were administered orally at a dose of 100 pg free base to 12 healthy male and female volunteers aged 18-45 years. Blood samples were withdrawn pre-dose and at 0.5, 1, 2, 3, 4, 5, 6, 8, 12, 16, 24, 32, and 48 hours post dose and submitted to analysis for the parent drug and prodrug using a validated LC-MS-MS assay. The following pharmacokinetic parameters derived from the plasma analytical data were determined;

Cmax Maximum measured concentration
Frel % Relative oral bioavailability of Guanfacine
t½ Elimination half life

The results are given in Table 7 below

TABLE 7
Guanfacine and compound 3 pharmacokinetic parameters following
administration to healthy human volunteers

| | | Cmax | AUC₀₋ₜ | t½ |
|---|---|---|---|---|
| Treatment | Analyte | (pg/mL) | (pg · h/mL) | (h) |
| Compound 3 | Compound 3 | 89 ± 38 | 92 ± 43 | 0.95 ± 0.19 |
| | | | | (n = 4) |
| Compound 3 | Guanfacine | 103 ± 77 | 2113 ± 524 | 17.2 ± 2.0 |
| Guanfacine | Guanfacine | 150 ± 37 | 3617 ± 933 | 15.8 ± 1.8 |

The data demonstrate absorption of the prodrug in man and subsequent cleavage to guanfacine. The relative bioavailability of guanfacine was 81% for compound 3 administration compared to guanfacine administration (adjusted for molecular weight, 100 μg of compound 3 free base contains 77.35 μg of guanfacine).

1. A compound of Formula (I) comprising: 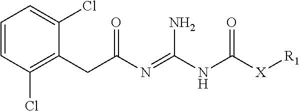
wherein
X is O or S;
R₁ is a C₁₋₂₀ substituted or unsubstituted alkyl, glycosyl, 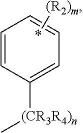
or a C₃₋₈ unsubstituted or substituted cycloalkyl;
R₂ is independently at each occurrence C₁₋₄ alkyl, C₁₋₄ alkoxy, halo, CN, NO₂, NH₂, SO₃H, OH, —CHO, —CO₂H,
or —CH₂CO₂H;
n is 0, 1, 2 or 3;
m is 0, 1, 2, 3, 4 or, 5; and
R₃ and R₄ are each independently selected at each occurrence from the group comprising: hydrogen, hydroxy, —CO₂H, methyl, and —NH₂.

2. The compound of claim 1, wherein X is O.

3. The compound of claim 1, wherein R₁ is a C₂₋₅ alkyl.

4. The compound of claim 1, wherein R₁ is ethyl, n-propyl, isopropyl, n-butyl, sec-butyl, isobutyl, neopentyl, 6-glucosyl, or benzyl.

5. The compound of claim 1, wherein R₁ is ethyl, n-propyl, isopropyl, n-butyl, or sec-butyl.

6. The compound of claim 1, wherein R₁ is a C₂₋₄ hydroxyalkyl.

7. The compound of claim 1, wherein R₁ is 2-hydroxyethyl, or 3-hydroxypropyl.

8. The compound of claim 1, wherein R₁ is phenyl acetic acid or meta-hydrobenzoic acid.

9. A compound of claim 1 selected from the group consisting of:

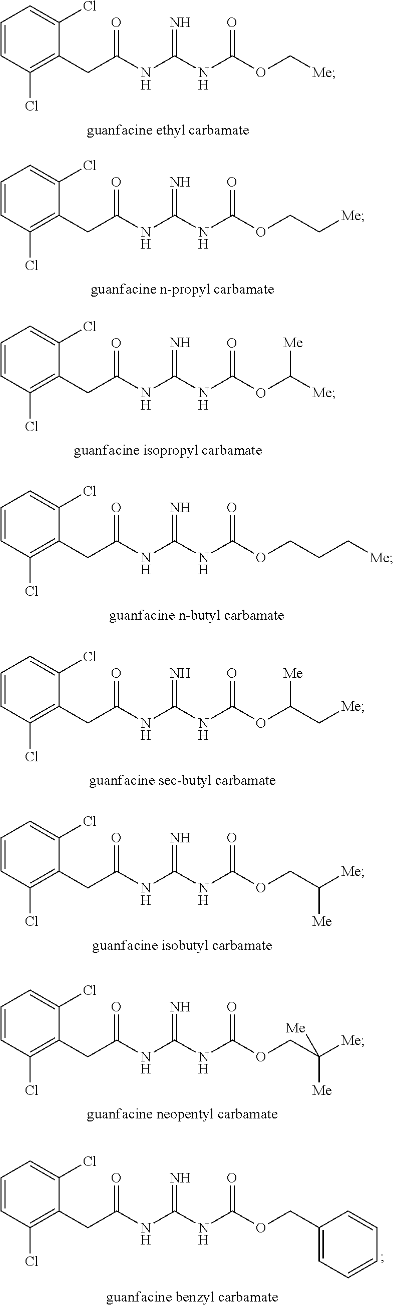

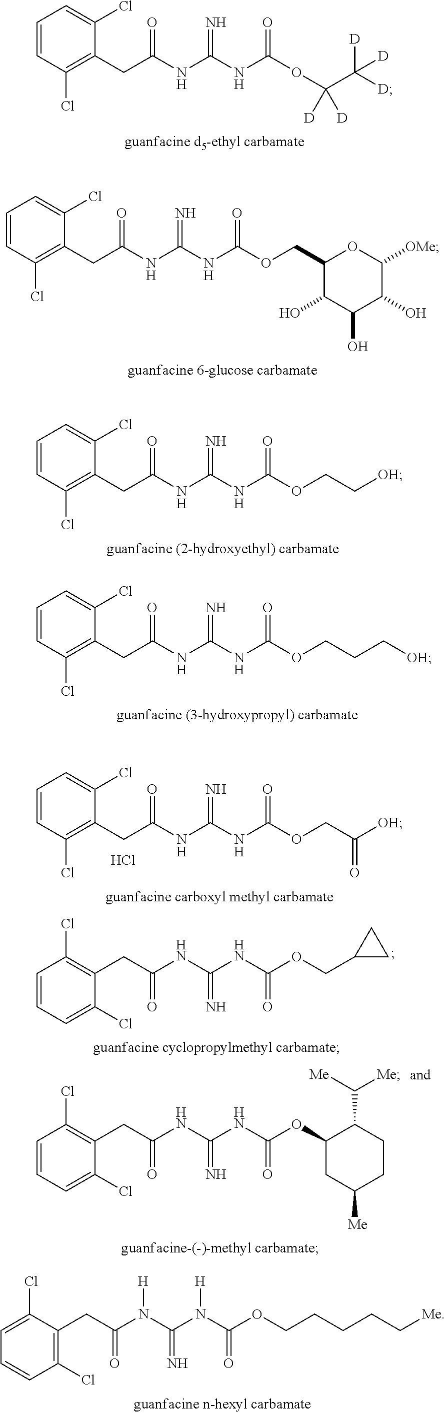

10. A compound of claim 1 selected from the group consisting of:

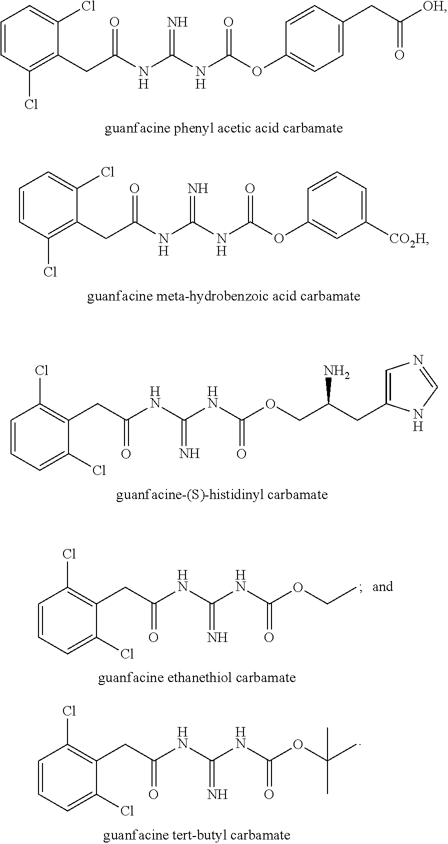

11. A method of treating a condition selected from the group consisting of: selected from the group consisting of: attention deficit hyperactivity disorder (ADHD), oppositional defiance disorder (ODD), a cardiovascular condition such as hypertension, neuropathic pain, cognitive impairment associated with schizophrenia (CMS), psychosis and working memory loss in the elderly, anxiety (including paediatric anxiety, PTSD, OCD, self injury), pruritis, addiction withdrawal, autism, chemotherapy induced mucositis, post traumatic stress syndrome or a disorder characterized by hot flushes in a mammal, comprising administering a guanfacine prodrug of claim 1 or a pharmaceutically acceptable salt thereof to a mammal in need thereof.

12. The method of claim 11, wherein the condition is attention deficit hyperactivity disorder (ADHD).

13. A method of reducing gastrointestinal side effects associated with guanfacine therapy in a mammal, comprising:
(a) forming a guanfacine prodrug of claim 1 or a pharmaceutically acceptable salt thereof; and
(b) administering the prodrug or a pharmaceutically acceptable salt thereof to a mammal in need thereof.

14. The method of claim 13, wherein the gastrointestinal side effects include constipation.

15. A method of treating an attention deficit hyperactivity disorder in a mammal, comprising administering a guanfacine prodrug of claim 1 or a pharmaceutically acceptable salt thereof to a mammal in need thereof.

16. A method of treating hypertension in a mammal, comprising administering a guanfacine prodrug of claim 1 or a pharmaceutically acceptable salt thereof to a mammal in need thereof.

17. The method of any of claims 13 to 16, wherein when ingested orally, the prodrug induces statistically significantly lower average effects on gut motility in the gastrointestinal environment than a non-prodrug guanfacine salt form.

18. The method of any of claims 13 to 16, wherein the prodrug or a pharmaceutically acceptable salt thereof is administered orally.

19. The method of any of claims 13 to 16, wherein the prodrug or a pharmaceutically acceptable salt thereof is administered in an amount of from about 1 to about 10 mg based on the amount of guanfacine in free base form.
